(19) United States
(12) Patent Application Publication
(10) Pub. No.: US 2023/0303722 A1
(43) Pub. Date: Sep. 28, 2023

(54) USE OF AN ACTIVATABLE, DE-ESTERIFIED FRUIT FIBER FOR THE MANUFACTURING OF PRODUCTS

(71) Applicant: Herbstreith & Fox GmbH & Co. KG Pektin-Fabriken, Neuenbürg (DE)

(72) Inventor: Gerhard F. Fox, Neuenbürg (DE)

(21) Appl. No.: 18/040,421

(22) PCT Filed: Aug. 4, 2021
(86) PCT No.: PCT/EP2021/071727

§ 371 Date: Feb. 2, 2023

(30) Foreign Application Priority Data
Aug. 5, 2020 (DE) 10 2020 120 605.4
Sep. 25, 2020 (DE) 10 2020 125 034.7

(51) Int. Cl. C08B 37/00; A23L 33/24; A23L 29/231; A23L 29/262; A23K 10/37; A23K 20/163; D01C 1/02; C08L 5/06

(52) U.S. Cl. CPC C08B 37/0045; A23L 33/24; A23L 29/231; A23L 29/262; A23K 10/37; A23K 20/163; D01C 1/02; C08B 37/0048; C08L 5/06; C08L 2205/16

(57) ABSTRACT

The present invention relates to the use of an activatable, de-esterified fruit fiber for preparing products in the food or non-food area. The invention also relates to products containing the activatable, de-esterified fruit fiber.

The present invention relates to the use of an activatable, de-esterified fruit fiber for the manufacturing of products in the food or non-food area. The invention also relates to products containing the activatable, de-esterified fruit fiber.

### BACKGROUND OF THE INVENTION

Dietary fibers are largely non-digestible food components, mostly carbohydrates, which predominantly exist in plant-based foods. For purposes of simplicity, fibers are divided into water-soluble fibers, such as pectin, and water-insoluble fibers, such as e.g. cellulose. Dietary fibers are considered to be an important part of the human diet.

Thus, the consumption of dietary fibers is considered to promote health. The use of fruit fibers, such as sugar beet, apple or citrus fibers, as dietary fibers in the production of food is gaining increasing importance. One reason is that fruit fibers are mixtures of insoluble fibers such as cellulose and soluble fibers such as pectin, resulting in an ideal spectrum of health-promoting activity. The functional properties of food products may be modified by use of fruit fibers like citrus fibers or apple fibers. Nowadays, fruit fibers are also employed in non-food products.

Thus, U.S. Pat. No. 5,964,983 teaches the use of a microfibrillar cellulose produced from sugar beets as a thickener for colors or drilling fluids. The method disclosed in U.S. Pat. No. 5,964,983, however, requires great effort since it comprises an acid or alkaline extraction, followed by an aqueous washing step, pressure homogenization, an ethanolic washing step and drying. In addition, the fiber properties are shown to change substantially, depending on the production method, consequently determining usability for the optimization of food or non-food products.

Thus, there is a demand for improved pectin-containing fruit fibers and the new or improved possibilities of their usage resulting therefrom.

The objective of the present invention thus is to provide an improvement or an alternative to the prior art.

### SUMMARY OF THE INVENTION

In a first aspect of the present invention, the objective is achieved by use of an activatable, de-esterified fruit fiber for manufacturing a product, selected from the group consisting of food products, feeding stuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesive, fertilizers, drugs, medical products, batteries, wherein the activatable, de-esterified fruit fiber has a content of water-soluble pectin of 10 wt % or less. Here, it is preferred that the activatable, de-esterified fruit fiber is an activatable, de-esterified citrus fiber or an activatable, de-esterified apple fiber.

The activatable, de-esterified fruit fiber advantageously has a content of water-soluble pectin of 10 wt % or less. The content of water-soluble pectin in an activatable, de-esterified citrus or apple fiber is 10 wt % or less, preferably between 2 wt % and 8 wt %, and particularly preferably of between 2 and 6 wt %. The content of water-soluble pectin in an activatable, de-esterified citrus or apple fiber can be, for example, 2 wt %, 3 wt %, 4 wt %, 5 wt %, 6 wt %, 7 wt %, 8 wt %, 9 wt % or 9.5 wt %.

The production method described in the following results in activatable, de-esterified fruit fibers with a large internal surface, which also increases water-binding capacity and contributes to a good viscosity formation. Especially in calcium-containing applications a significant gelation can additionally be observed.

These fibers are activatable fibers that display satisfactory firmness due to the partial activation of the manufacturing process. However, to obtain optimal rheological properties like viscosity, gelation or texturing, additional shear forces are required in application for the user. These are thus partially activated fibers which can, however, be further activated. The term “partially activated fibers” is thus synonymous with the term “activatable fibers” in the context of the present application.

Additionally, the activatable, de-esterified fruit fiber used according to the invention has a more potent effectiveness. Compared to modified starch, less than half the amount needs to be employed to produce a fat-containing cream with comparable baking stability.

The activatable de-esterified, pectin-converted fruit fiber (i.e., water-soluble pectin content less than or equal to 10 wt %) and low methoxyl fruit fiber obtained by the process described herein is in the context of the invention also referred to as “de-esterified fruit fiber”.

The inventors have found the citrus fibers produced with the method described below to exhibit good rheological characteristics. The fibers used according to the invention can be easily rehydrated in calcium-free water, and the advantageous rheological properties remain even after rehydration.

The inventors have found the activatable, de-esterified fruit fibers according to the invention to be to a high degree without taste and smell, which makes them advantageously usable for the application in the food industry. The inherent flavor of the other ingredients is not masked and can therefore develop optimally.

The activatable, de-esterified fruit fibers are extracted from fruits and thus represent natural ingredients with known beneficial properties.

Vegetable processing residues such as apple pomace or citrus pomace can be used as raw material in the production process described below. These processing residues are inexpensive, are available in sufficient quantities and offer a sustainable and ecologically sensible source for the fruit fibers that can be used according to the invention.

Fruit fibers are well-established and accepted in the food industry so that respective compositions can be immediately used without lengthy admission procedures, even internationally.

### DETAILED DESCRIPTION OF THE INVENTION

In a second aspect, the invention relates to the use of an activatable, de-esterified fruit fiber in the construction area, in extraction by drilling of boreholes and in the agricultural field, characterized in that the activatable, de-esterified fruit fiber has a content of water-soluble pectin of 10 wt % or less. Here, it is preferred that the activatable, de-esterified fruit fiber is an activatable, de-esterified citrus fiber or an activatable, de-esterified apple fiber.

In the uses taught above, the activatable, de-esterified fruit fiber used according to the invention can have one or more of the following functions: as a foaming agent, a whipping agent, a release agent, a free flow agent, a stabilizer, an emulsifier, a carrier, a filler, a texturing agent, a thickener, a gelling agent, a solidifying agent, a dietary fiber, a reinforcing agent, a humectant, a filter aid, an egg substitute, a glazing agent, an improving agent for freeze-thaw stability and an improving agent for baking stability.

The invention relates to the use of an activatable, de-esterified fruit fiber. Such an activatable, de-esterified fruit fiber can be obtained from pomace such as apple, or citrus pomace, which is digested by incubation of an aqueous suspension of citrus or apple pomace as starting material.

The activatable pectin-containing fruit fiber used according to the invention contains water-soluble pectin in addition to the insoluble fiber-bound pectin (also known as protopectin). Protopectins are insoluble pectins and presumably not pure homoglycans.

In protopectin, the polygalacturonic acid chains are interconnected by complex bonds with divalent cations, via ferulic acid groups and borate complexes, and via glycosidic bonds with neutral sugar side chains, which may consist of arabinose, galactose, xylose, mannose and traces of fucose. Since the citrus fiber, as explained above, also contains water-soluble pectin, it is also referred to as “pectin-containing citrus fiber” in the context of the invention.

The Activatable, De-Esterified Fruit Fiber

The activatable, de-esterified fruit fiber is preferably an activatable, de-esterified citrus fiber or an activatable, de-esterified apple fiber.

In detail:

The Activatable, De-Esterified Citrus Fiber

The activatable, de-esterified citrus fiber used according to the invention has a water-soluble pectin content of 10 wt % or less, the pectin having a degree of esterification of less than 50% and thus being a low methoxyl pectin. This activatable low methoxyl citrus fiber low in pectin is also briefly referred to as “de-esterified citrus fiber” in the context of the invention. This de-esterified citrus fiber is preferably obtainable by, or obtained by, the process described herein.

The de-esterified citrus fiber used according to the invention advantageously has a water-soluble pectin content of between 2 wt % and 8 wt %, and particularly preferably of between 2 and 6 wt %. The content of water-soluble pectin in an activatable, de-esterified citrus fiber can be, for example, 2 wt %, 3 wt %, 4 wt %, 5 wt %, 6 wt %, 7 wt %, 8 wt %, 9 wt % or 9.5 wt %.

The de-esterified citrus fiber exhibits advantageous characteristics in terms of texturization and viscosification behavior, which can be read off from the yield point and dynamic Weissenberg number, respectively. Accordingly, the de-esterified citrus fiber may exhibit one or more of the following characteristics with respect to yield point and dynamic Weissenberg number, and advantageously may satisfy all of these characteristics.

In one embodiment, the de-esterified citrus fiber in a 2.5 wt % aqueous suspension has a yield point II (rotation) of 0.1-1.5 Pa, advantageously of 0.4-1.0 Pa, and particularly advantageously of 0.6-0.8 Pa.

In one embodiment, the de-esterified citrus fiber in a 2.5 wt % aqueous suspension has a yield point II (cross over) of 0.1-1.0 Pa, advantageously of 0.2-0.7 Pa, and particularly advantageously of 0.3-0.5 Pa.

In one embodiment, the de-esterified citrus fiber in a 2.5 wt % aqueous dispersion has a yield point I (rotation) of 3.0-7.0 Pa, advantageously of 4.0-5.5 Pa, and particularly advantageously of 4.3-5.3 Pa.

In one embodiment, the de-esterified citrus fiber in a 2.5 wt % aqueous dispersion has a yield point I (cross over) of 4.0-7.0 Pa, advantageously of 4.5-6.5 Pa, and particularly advantageously of 5.0-6.0 Pa.

Advantageously, the de-esterified citrus fiber in a 2.5 wt % aqueous suspension has a dynamic Weissenberg number of 7.0-10.0 Pa, advantageously of 7.5-9.5 Pa, and particularly advantageously of 8.1-9.1 Pa.

Advantageously, the de-esterified citrus fiber in a 2.5 wt % aqueous dispersion has a dynamic Weissenberg number of 7.5-10.0 Pa, advantageously of 8.0-9.5 Pa, and particularly advantageously of 8.3-9.3 Pa.

For the de-esterified citrus fiber, the features of the above-described characteristics with respect to yield point and dynamic Weissenberg number may also be combined in any permutation, if desired. Thus, in a particular embodiment, the de-esterified citrus fiber used according to the invention may have all the characteristics with respect to yield point and dynamic Weissenberg number, wherein said de-esterified citrus fiber preferably is obtainable by the present process or obtained thereby.

To determine yield point I (rotation), yield point I (cross over), and dynamic Weissenberg number in a 2.5 wt % aqueous dispersion, the de-esterified citrus fiber is dispersed as a 2.5 wt % aqueous solution according to the method disclosed in the examples, and the measurement is made after 1 h at 20° C.

To determine yield point II (rotation), yield point II (cross over) and dynamic Weissenberg number in a 2.5 wt % aqueous suspension, the de-esterified citrus fiber is suspended as a 2.5 wt % aqueous solution according to the method disclosed in the examples, measurement is carried out after 1 h at 20° C.

In an advantageous embodiment, the de-esterified citrus fiber has, in a 4 wt % aqueous suspension, a firmness of between 60 g and 240 g, preferably of between 120 g and 200 g, and particularly preferably of between 140 g and 180 g.

Preferably, the de-esterified citrus fiber has a viscosity of between 550 and 850 mPas, preferably of between 600 and 800 mPas, and particularly preferably of between 650 and 750 mPas, wherein the de-esterified citrus fiber is dispersed in water as a 2.5 wt % solution and the viscosity is measured with a shear rate of 50 s⁻¹ at 20° C.

For determining viscosity, the citrus fiber is dispersed in demineralized water with the method disclosed in the examples as a 2.5 wt % solution, and viscosity is determined at 20° C. and four shearing sections (first and third section =constant profile; second and fourth section =linear ramp; each measurement at a shearing speed of 50 s⁻¹) (rheometer; Physica MCR series, measuring bob CC25 [corresponding to Z3 DIN], Anton Paar company, Graz, Austria). The advantage of a de-esterified citrus fiber with such a high viscosity is that a lower amount of fiber is necessary for thickening the final product. In addition, the fiber thus creates a creamy texture.

The de-esterified citrus fiber advantageously has a water binding capacity of more than 24 g/g, preferably of more than 26 g/g, particularly preferably of more than 28 g/g, and especially preferably of between 28 and 32 g/g. Such an advantageously high-water binding capacity leads to a high viscosity and consequently also to a lower fiber consumption with a creamy texture.

According to one embodiment, the de-esterified citrus fiber has a moisture of less than 15%, preferably less than 10% and particularly preferably less than 8%.

It is also preferable for the de-esterified citrus fiber to have, in a 1.0% aqueous solution, a pH value of 5.0 to 6.0 and preferably of 5.2 to 5.7.

The de-esterified citrus fiber advantageously has a particle size in which at least 90% of the particles are smaller than 450 μm, preferably smaller than 350 μm and particularly preferably smaller than 250 μm.

In one advantageous embodiment, the de-esterified citrus fiber has a lightness value of L*>84, preferably L*>86 and particularly preferably L*>88. This means that the citrus fibers are virtually colorless and do not cause any significant discoloration of the products when used in food products.

Advantageously, the de-esterified citrus fiber has a dietary fiber content of 80 to 95%.

Due to the acidic extraction step, the pectin content of the citrus fiber has been strongly reduced so that de-esterified citrus fiber has 10 wt % or less of water-soluble pectin. Preferably, the de-esterified citrus fiber has less than 8 wt % and particularly preferably less than 6 wt % of water-soluble pectin. The de-esterified citrus fiber advantageously has a content of water-soluble pectin of between 2 wt % and 8 wt % and particularly preferably of between 2 and 6 wt %. The content of water-soluble pectin in the de-esterified citrus fiber can be, for instance, 2 wt %, 3 wt %, 4 wt %, 5 wt %, 6 wt %, 7 wt %, 8 wt %, 9 wt % or 9.5 wt %.

This residual pectin is low methoxyl pectin due to the subsequent enzymatic de-esterification step. According to the invention, a low methoxyl pectin is a pectin which has a degree of esterification of less than 50%. The degree of esterification is the percentage of carboxylic groups in the galacturonic acid chains of the pectin which are present in esterified form, e. g. as methyl esters. The degree of esterification can be determined with the method according to JECFA (Monograph 19-2016, Joint FAO/WHO Expert Committee on Food Additives). The combination of de-pectinization and de-esterification thus yields the citrus fiber that can be used according to the invention, which is referred to in the context of the invention as “de-esterified citrus fiber”.

The de-esterified citrus fiber used according to the invention is preferably present in powder form. The advantage is that in this manner, there is a formulation with low weight and long shelf life which is also easy to employ in process technology. This formulation is only made possible by the citrus fiber used according to the invention which, other than modified starches, does not tend to lump formation when it is dissolved in liquids.

The De-Esterified Apple Fiber

The activatable, de-esterified apple fiber used according to the invention has a water-soluble pectin content of 10 wt % or less, the pectin having a degree of esterification of less than 50% and thus being a low methoxyl pectin. This activatable low methoxyl apple fiber low in pectin is also briefly referred to as “de-esterified apple fiber” in the context of the invention. This de-esterified apple fiber is preferably obtainable by, or obtained by, the process described herein.

The de-esterified apple fiber used according to the invention advantageously has a water-soluble pectin content of between 2 wt % and 8 wt %, and particularly preferably of between 2 and 6 wt %. The content of water-soluble pectin in an activatable, de-esterified apple fiber can be, for example, 2 wt %, 3 wt %, 4 wt %, 5 wt %, 6 wt %, 7 wt %, 8 wt %, 9 wt % or 9.5 wt %.

The de-esterified apple fiber exhibits advantageous characteristics in terms of texturization and viscosification behavior, which can be read off from the yield point and dynamic Weissenberg number, respectively. Accordingly, the de-esterified apple fiber may exhibit one or more of the following characteristics with respect to yield point and dynamic Weissenberg number, and advantageously may satisfy all of these characteristics.

In one embodiment, the de-esterified apple fiber in a 2.5 wt % aqueous suspension has a yield point II (rotation) of 0.1-1.5 Pa, advantageously of 0.4-1.0 Pa, and particularly advantageously of 0.6-0.8 Pa.

In one embodiment, the de-esterified apple fiber in a 2.5 wt % aqueous suspension has a yield point II (cross over) of 0.1-1.0 Pa, advantageously of 0.2-0.7 Pa, and particularly advantageously of 0.3-0.5 Pa.

In one embodiment, the de-esterified apple fiber in a 2.5 wt % aqueous dispersion has a yield point I (rotation) of 3.0-7.0 Pa, advantageously of 4.0-5.5 Pa, and particularly advantageously of 4.3-5.3 Pa.

In one embodiment, the de-esterified apple fiber in a 2.5 wt % aqueous dispersion has a yield point I (cross over) of 4.0-7.0 Pa, advantageously of 4.5-6.5 Pa, and particularly advantageously of 5.0-6.0 Pa.

Advantageously, the de-esterified apple fiber in a 2.5 wt % aqueous suspension has a dynamic Weissenberg number of 7.0-10.0 Pa, advantageously of 7.5-9.5 Pa, and particularly advantageously of 8.1-9.1 Pa.

Advantageously, the de-esterified apple fiber in a 2.5 wt % aqueous dispersion has a dynamic Weissenberg number of 7.5-10.0 Pa, advantageously of 8.0-9.5 Pa, and particularly advantageously of 8.3-9.3 Pa.

For the de-esterified apple fiber, the features of the above-described characteristics with respect to yield point and dynamic Weissenberg number may also be combined in any permutation, if desired. Thus, in a particular embodiment, the de-esterified apple fiber used according to the invention may have all the characteristics with respect to yield point and dynamic Weissenberg number, wherein said de-esterified apple fiber preferably is obtainable by the present process or obtained thereby.

To determine yield point I (rotation), yield point I (cross over), and dynamic Weissenberg number in a 2.5 wt % aqueous dispersion, the de-esterified apple fiber is dispersed as a 2.5 wt % aqueous solution according to the method disclosed in the examples, and the measurement is made after 1 h at 20° C.

To determine yield point II (rotation), yield point II (cross over) and dynamic Weissenberg number in a 2.5 wt % aqueous suspension, the de-esterified apple fiber is suspended as a 2.5 wt % aqueous solution according to the method disclosed in the examples, measurement is carried out after 1 h at 20° C.

In an advantageous embodiment, the de-esterified apple fiber has, in a 4 wt % aqueous suspension, a firmness of between 60 g and 240 g, preferably of between 120 g and 200 g, and particularly preferably of between 140 g and 180 g.

Preferably, the de-esterified apple fiber has a viscosity of between 550 and 850 mPas, preferably of between 600 and 800 mPas, and particularly preferably of between 650 and 750 mPas, wherein the de-esterified apple fiber is dispersed in water as a 2.5 wt % solution and the viscosity is measured with a shear rate of 50 s⁻¹ at 20° C.

For determining viscosity, the apple fiber is dispersed in demineralized water with the method disclosed in the examples as a 2.5 wt % solution, and viscosity is determined at 20° C. and four shearing sections (first and third section =constant profile; second and fourth section =linear ramp; each measurement at a shearing speed of 50 s⁻¹) (rheometer; Physica MCR series, measuring bob CC25 [corresponding to Z3 DIN], Anton Paar company, Graz, Austria). The advantage of a de-esterified apple fiber with such a high viscosity is that a lower amount of fiber is necessary for thickening the final product. In addition, the fiber thus creates a creamy texture.

The de-esterified apple fiber advantageously has a water binding capacity of more than 24 g/g, preferably of more than 26 g/g, particularly preferably of more than 28 g/g, and especially preferably of between 28 and 32 g/g. Such an advantageously high-water binding capacity leads to a high viscosity and consequently also to a lower fiber consumption with a creamy texture.

According to one embodiment, the de-esterified apple fiber has a moisture of less than 15%, preferably less than 10% and particularly preferably less than 8%.

It is also preferable for the de-esterified apple fiber to have, in a 1.0% aqueous solution, a pH value of 5.0 to 6.0 and preferably of 5.2 to 5.7.

The de-esterified apple fiber advantageously has a particle size in which at least 90% of the particles are smaller than 450 μm, preferably smaller than 350 μm and particularly preferably smaller than 250 μm.

In one advantageous embodiment, the de-esterified apple fiber has a lightness value of L*>60, preferably L*>61 and particularly preferably L*>62. This means that the apple fibers are virtually colorless and do not cause any significant discoloration of the products when used in food products.

Advantageously, the de-esterified apple fiber has a dietary fiber content of 80 to 95%.

Due to the acidic extraction step, the pectin content of the apple fiber has been strongly reduced so that de-esterified apple fiber has 10 wt % or less of water-soluble pectin. Preferably, the de-esterified apple fiber has less than 8 wt % and particularly preferably less than 6 wt % of water-soluble pectin. The de-esterified apple fiber advantageously has a content of water-soluble pectin of between 2 wt % and 8 wt % and particularly preferably of between 2 and 6 wt %. The content of water-soluble pectin in the de-esterified apple fiber can be, for instance, 2 wt %, 3 wt %, 4 wt %, 5 wt %, 6 wt %, 7 wt %, 8 wt %, 9 wt % or 9.5 wt %.

This residual pectin is low methoxyl pectin due to the subsequent enzymatic de-esterification step. According to the invention, a low methoxyl pectin is a pectin which has a degree of esterification of less than 50%. The degree of esterification is the percentage of carboxylic groups in the galacturonic acid chains of the pectin which are present in esterified form, e. g. as methyl esters. The degree of esterification can be determined with the method according to JECFA (Monograph 19-2016, Joint FAO/WHO Expert Committee on Food Additives). The combination of de-pectinization and de-esterification thus yields the apple fiber that can be used according to the invention, which is referred to in the context of the invention as “de-esterified apple fiber”.

The de-esterified apple fiber used according to the invention is preferably present in powder form. The advantage is that in this manner, there is a formulation with low weight and long shelf life which is also easy to employ in process technology. This formulation is only made possible by the apple fiber used according to the invention which, other than modified starches, does not tend to lump formation when it is dissolved in liquids.

Production of the Activatable, De-Esterified Fruit Fiber

The activatable, de-esterified fruit fiber is preferably a de-esterified citrus fiber or a de-esterified apple fiber and is obtainable by a method comprising the following steps:
(a) providing a raw material which contains the cell wall material of an edible fruit, preferably a citrus fruit or an apple fruit;
(b) disintegrating the raw material by incubation of an aqueous suspension of the raw material at an acidic pH value;
(c) one- or multi-stage separation of the disintegrated material in step (b) from the aqueous suspension and optionally a separation of larger particles by a classification process;
(d) Enzymatic or acidic de-esterification of the separated, optionally classified material from step (c);
(e) In the case of a material classified according to step (c), concentration of the de-esterified material from step (d) by ultrafiltration or evaporation of the liquid;
(f) washing the de-esterified material from step (d) or the concentrated material from step (e) at least twice with an organic water-miscible solvent and subsequently separating the washed material respectively from the organic water-miscible solvent;
(g) optionally additionally removing the organic water-miscible solvent by contacting the washed material from step (f) with water vapor;
(h) drying the material from step (f) or (g), comprising drying at normal pressure or vacuum drying to obtain the de-esterified fruit fiber.

A fruit fiber according to the invention is a plant fiber, i.e. a fiber isolated from a non-lignified plant cell wall and consists mainly of cellulose, and which is hereby isolated from a fruit. A fruit is understood herein to be the totality of the organs of a plant that arise from a flower, including both the classical fruit fruits and fruit vegetables.

In a preferred embodiment, said fruit fiber is selected from the group consisting of citrus fiber, apple fiber, sugar beet fiber, carrot fiber and pea fiber, wherein the plant fiber is preferably a fruit fiber and particularly preferably a citrus fiber or an apple fiber.

An “apple fiber” in the sense of the application is a component consisting mainly of fibers, which is isolated from a non-lignified cellular wall of an apple and consists mainly of cellulose. In a sense, the term “fiber” is a misnomer since macroscopically, the apple fibers do not appear as fibers but as a powdery product.

Other components of the apple fiber are, among others, hemicellulose and pectin.

The apple fiber can be obtained from all cultivated apples (malus domesticus) known to the person skilled in the art. As starting material, advantageously processing residues of apples can be employed. The starting material may be apple peel, apple core, apple seeds, fruit flesh or a combination thereof. Preferably, apple pomace is used as the raw material, i. e. the press residues of apples, which typically also contain the above-mentioned components in addition to the peels.

A “citrus fiber” in the sense of the application is a component consisting mainly of fibers, which is isolated from a non-lignified cellular wall of a citrus fruit and consists mainly of cellulose. In a sense, the term “fiber” is a misnomer since macroscopically, the citrus fibers do not appear as fibers but as a powdery product. Other components of the citrus fiber are, among others, hemicellulose and pectin. The citrus fiber can advantageously be obtained from citrus pulp, citrus peel, citrus vesicle, segment membranes or a combination thereof.

For the production of a de-esterified citrus fiber, citrus fruits, and preferably processing residues of citrus fruits, can be employed as raw material. The raw material may be citrus peel (albedo and/or flavedo), citrus vesicles, segment membranes or a combination thereof. Preferably, citrus pulp is used as the raw material, i. e. the press residues of citrus fruits, which typically also contain the fruit flesh in addition to the peels.

All citrus fruits known to the skilled person can be used as citrus fruits in this context. The following are listed by way of example in a non-restrictive manner: Mandarin (Citrus reticulata), Clementine (Citrus×aurantium Clementine group, Syn.: Citrus clementina), Satsuma (Citrus xaurantium Satsuma group, Syn.: Citrus unshiu), Mangshan (Citrus mangshanensis), Orange (Citrus xaurantium orange group, Syn.: Citrus sinensis), Bitter Orange (Citrus xaurantium bitter orange group), Bergamot (Citrus xlimon bergamot group, Syn.: Citrus bergamia), grapefruit (Citrus maxima), grapefruit (Citrus xaurantium grapefruit group, syn.: Citrus paradisi) pomelo (Citrus xaurantium pomelo group), true lime (Citrus xaurantiifolia), common lime (Citrus xaurantiifolia, syn. Citrus latifolia), kaffir lime (Citrus hystrix), Rangpur lime (Citrus xjambhiri), lemon (Citrus xlimon lemon group), citron (Citrus medica) and kumquats (Citrus japonica, Syn.: Fortunella). Preferred among these are orange (Citrus xaurantium orange group, syn.: Citrus sinensis) and lemon (Citrus xlimon lemon group).

The acidic disintegration in step (b) of the method is used to remove pectin by converting the protopectin into soluble pectin and at the same time activating the fiber by enlargement of the interior surface. Furthermore, the raw material is thermally comminuted by the disintegration. Due to acidic incubation in the aqueous environment, with the application of heat, it disintegrates into fruit fibers. In this way, thermal comminution is achieved; mechanical comminution is not necessary within the framework of this production method. This is a substantial advantage over conventional fiber production methods which, in contrast, require a shearing step (for instance [high] pressure homogenization) in order to obtain a fiber with sufficient rheological properties.

During the disintegration, the raw material is an aqueous suspension. A suspension according to the invention is a heterogeneous mixture of a liquid and solids (raw material particles) finely distributed therein. Since the suspension tends to sedimentation and separation of phases, the particles are suitably kept in suspension by shaking or stirring. That is, there is no dispersion, which would mean that the particles are mechanically comminuted (shearing) so as to be finely dispersed.

To achieve an acidic pH value in step (b), the person skilled in the art may employ all acids or acidic buffering solutions that are known to him. For example, an organic acid such as citric acid may be employed.

Alternatively, or in combination, a mineral acid may be used. Some examples are sulfuric acid, hydrochloric acid, nitric acid or sulfurous acid. Preferably, nitric acid or sulfuric acid is employed.

In acidic disintegration according to step (b) of the method, the pH value of the suspension is between pH=0.5 and pH=4.0, preferably between pH=1.0 and pH=3.5 and particularly preferably between pH=1.5 and pH=3.0. For example, the optional acidic disintegration can be performed at a pH of 0.75, 1.0, 1.25, 1.5, 1.75, 2.0, 2.25, 2.5, 2.75, 3.0, 3.25, 3.5 or 3.75.

Advantageously, the liquid for producing the aqueous suspension of the acidic disintegration in step (b) consists of more than 50 vol %, preferably more than 60, 70, 80 or even 90 vol %, of water. In a preferred embodiment, the liquid contains no organic solvent and in particular no alcohol. Thus, the process is a water-based acidic extraction.

In the acidic disintegration, the incubation takes place at a temperature between 60° C. and 95° C., preferably between 70° C. and 90° C., and particularly preferably between 75° C. and 85° C. For example, the acidic disintegration can be performed at a temperature of 61° C., 62° C., 63° C., 64° C., 65″C, 66° C., 67° C., 68° C., 69° C., 70° C., 71° C., 72° C., 73° C., 74° C., 75° C., 76° C., 77° C., 78° C., 79° C., 80° C., 81° C., 82° C., 83° C. or 84° C.

In the acidic disintegration, the incubation takes place over 60 min to 8 hours, and preferably over 2 to 6 hours. For example, the acidic disintegration can be performed over a period of 1.5 h, 2.0 h, 2.5 h, 3.0 h, 3.5 h, 4.0 h, 4.5 h, 5.0 h, 5.5 h, 6.0 h, 6.5 h, 7.0 h or 7.5 h.

In acidic disintegration, the aqueous suspension suitably has a dry mass of between 0.5 wt % and 5 wt % preferably of between 1 wt % and 4 wt %, and particularly preferably of between 1.5 wt % and 3 wt %. For example, the dry weight may be 0.5, 0.75, 1.0, 1.25, 1.5, 1.75, 2.0, 2.25, 2.5, 2.75, 3.0, 3.75, 4.0, 4.25, 4.5 or 4.75 wt % for the acidic disintegration.

During the disintegration, the aqueous suspension is stirred or shaken. This is preferably done in a continuous manner so that the particles in the suspension are kept in suspension.

In step (c) of the method, the disintegrated material is separated from the aqueous solution and thus recovered. This separation takes place in one or more steps.

Advantageously, the disintegrated material is subjected to multi-step solid-liquid-separation. Preferably, the first separation is done by means of decanters and the second one by means of separators. Here, based on the aqueous fiber suspension the solid is separated independently of the particle size from the liquid, contrary to a classification process.

Optionally, larger particles can also be separated in step (c). This is preferably done by a classification process. In the context of the present invention, a classification process is understood to mean the separation of a disperse solid mixture into fractions according to particle size. In the simplest case, two fractions are produced here, but two or three particle fractions with a defined particle distribution can also be produced as part of the classification process. The classic process here is sieving.

Here, particularly advantageous is the separation of particles with a particle size of more than 500 μm, preferably more than 400 μm and most preferably more than 350 μm. The separation is advantageously carried out with a straining machine or a sieving drum. This removes both coarse particulate impurities from the raw material and insufficiently digested material. The need to perform this optional separation step depends on the firmness of the fiber material to be pulped. While it is regularly necessary for citrus fibers, apple fibers disintegrate into fine fibers during the acid hydrolysis step, so that this separation step can be regularly omitted.

In one embodiment, the classification process uses a sieve drum in which the mesh size is set so that the larger particles to be separated accrue in the sieve overflow and the smaller particles to be processed further accrue in the sieve pass. The sieve overflow can be fed to a new sieving process for subsequent cleaning. In this case, the sieve overflow is preferably conveyed to the second sieve drum by a washing screw. In one embodiment, the sieve overflow is washed by water or an aqueous buffer during transport in the washing screw so that, for example, adhering pectin can be removed.

Alternatively, wet sieving can be used as the sieving process.

The skilled person is familiar with numerous sieving machines for carrying out classification processes, which he will select according to the fiber particle size present and the fact that a moist or wet material is present. Examples of sieving machines include cantilever sieving machines, elliptical sieving machines, eccentric sieving machines, linear sieving machines, throw sieving machines, planar sieving machines, beater sieving machines, air jet sieving machines, and eddy current machines.

The classification process in step (c) can be carried out during the single or multi-stage separation of the disintegrated material from the aqueous liquid, before this separation, or after the separation from the aqueous liquid.

If the classification process is carried out after the single or multi-stage separation of the disintegrated material from the aqueous liquid, it must be resuspended with an aqueous solution to produce an aqueous suspension with reduced dry substance content, as required by the corresponding classification process.

In the context of the invention, the “aqueous solution” is understood to be the aqueous liquid used for resuspension and incubation. The mixture of this aqueous solution and the digested material is referred to as the “incubation mixture”.

Advantageously, resuspension is carried out using water as the aqueous solution. Particularly advantageous here is the use of an aqueous buffer solution, so that a pH of 3.5 to 5.5, and advantageously of 4.0 to 5.0, results in the suspension. This pH increase based on the strongly acidic pH value of the acidic disintegration is carried out to provide an optimum pH for the subsequent enzymatic de-esterification. Alternatively, the adjustment of the pH to the value of 3.5 to 5.5, and advantageously from 4.0 to 5.0 can also be carried out after resuspension.

For this partial neutralization NaOH, KOH or a sodium- or potassium-salt such as sodium carbonate, sodium hydrogen carbonate, potassium carbonate, or potassium hydrogen carbonate can be used.

Alternatively, a saline solution with an ionic strength of I<0.2 mol/l can be used as the aqueous solution.

The resuspension is advantageously carried out at a temperature between 10° C. and 70° C., preferably between 20° C. and 60° C., and particularly preferably between 30° C. and 50° C.

When resuspending to carry out the classification process, advantageously so much liquid is added that the dry matter lies between 0.5 wt % and 5 wt %.

Advantageously, resuspension is carried out with mechanical agitation of the incubation mixture. This is more conveniently done by stirring or shaking the wash mixture.

After the solid-liquid separation and the optional separation of larger particles, the separated, optionally classified material from step (c) is incubated with a pectin methyl esterase or subjected to acidic de-esterification according to step (d).

In case the material has been subjected to a classification process in step (c) in addition to the solid-liquid separation, it is present as a suspension with low dry matter content (<12%dm) due to the resuspension required for this purpose. Enzyme treatment is then expediently carried out in a stirred tank.

In the case that no classification process was used in step (c), the material is present as a suspension with a high dry substance content (>12%dm) as a result of the solid-liquid separation. In this case, the enzyme treatment is expediently carried out in a thick matter reactor.

The following table shows some examples of commercially available PME with their reaction optima:

| Product name | Manufacturer | Optima |
|---|---|---|
| Rapidase PEP | DSM | pH = 4-5; T = 50° C. |
| Pectinase 872 L | Biocatalysts | pH = 4-5; T = 30-50° C. |
| Pectinesterase 1508/14 | Erbslöh | pH = 4-5; T = 30-50° C. |

A pectin methylesterase (abbreviation: PME, EC 3.1.1.11, also: pectin demethoxylase, pectin methoxylase) is a commonly found enzyme in the cell wall in all higher plants and some bacteria and fungi, which cleaves the methyl esters of pectins, forming poly-galacturonic acid and releasing methanol. PME has been isolated in many isoforms, all of which can be used for enzymatic de-esterification according to the invention. preferred pectin methylesterases have a pH optimum between 2 and 5 and a temperature optimum at 30 to 50° C., although depending on the enzyme, appreciable enzyme activity can be observed from as low as 15° C.

The duration of the incubation with the pectin methylesterase is between 1 hour and 10 hours, preferably between 2 hours and 5 hours.

During acidic de-esterification in step (d) of the process, the pH of the suspension is between pH=1.0 and pH=2.0. The acidic de-esterification according to step (e) can be carried out, for example, at a pH of 1.1, 1.2, 1.3, 1.4, 1.5, 1.6, 1.7, 1.8 or 1.9.

Advantageously, the liquid for producing the aqueous suspension of the acidic de-esterification in step (d) consists of more than 50 vol %, preferably more than 60, 70, 80 or even 90 vol %, of water. In a preferred embodiment, the liquid contains no organic solvent and in particular no alcohol. Thus, the process is a water-based acidic de-esterification.

The acidic de-esterification according to step (d) is carried out at a temperature of between 30° C. and 60° C. For example, it can be carried out at a temperature of 35° C., 40° C., 45° C., 55° C. or 60° C.

In the acidic de-esterification according to step (d), the incubation is carried out for a period of between 30 min to 10 days and preferably between 2 h and 6 hours. For example, the acid digestion according to step (c) can be carried out over a time period of 1.5 h, 2.0 h, 2.5 h, 3.0 h, 3.5 h, 4.0 h, 4.5 h, 5.0 h, 5.5 h or 6.0 h.

After incubation with the enzyme or acidic de-esterification according to step (d), the de-esterified material is concentrated in step (e) if a classification process has been carried out beforehand and the result of step (c) and step (d) is a suspension with a low dry substance content (<12%dm).

This concentration can be carried out, for example, by ultracentrifugation or evaporation of the aqueous liquid. This concentration is designed in such a way that the pectin is retained in the material, i.e. is essentially not separated by the concentration.

In step (f), a washing step is then performed with a water-miscible organic water-miscible solvent. This involves washing at least twice with the organic water-miscible solvent.

A solvent means here at least one solvent, so that two, three or more organic water-miscible solvents may also be contained.

The washing liquid preferably comprises more than 70 vol %, particularly preferably more than 80 vol %, and especially preferably more than 85 vol % of the water-miscible organic solvent. For example, the washing liquid may comprise 80%, 81%, 82%, 83%, 84%, 85%, 86%, 87%, 88%, 89%, 90%, 91%, 92%, 93%, 94%, 95%, 96%, 97%, 98%, 99% or 99.5% of water-miscible organic solvent, the percentages representing volume percentages. In an alternative embodiment, the washing liquid comprises the organic water-miscible solvent.

The further ingredient which complements this organic water-miscible solvent to 100% is expediently water or an aqueous buffer.

Water-miscible, thermally stable, volatile solvents containing only carbon, hydrogen and oxygen, such as alcohols, ethers, esters, ketones and acetals, are particularly suitable for carrying out the present process. Preferably, ethanol, n-propanol, isopropanol, methyl ethyl ketone, 1,2-butanediol-1-methyl ether, 1,2-propanediol-1-n-propyl ether or acetone are used.

An organic solvent is described as “water-miscible” if it is present as a single-phase liquid in a 1:20 (v/v) mixture with water.

In general, expediently solvents are used which are at least 10% water-miscible, have a boiling point below 100° C. and/or fewer than 10 carbon atoms.

The water-miscible organic solvent is preferably an alcohol, advantageously selected from the group consisting of methanol, ethanol and isopropanol. In a particularly preferred manner, it is isopropanol.

The washing step is carried out at a temperature between 40° C. and 75° C., preferably between 50° C. and 70° C. and particularly preferably 60° C. and 65° C.

The contacting with the water-miscible organic solvent in step (f) takes place over a period of time of between 60 min and 10 h, and preferably between 2 h and 8 h.

Each washing step with the water-miscible organic solvent comprises bringing the material into contact with the water-miscible organic solvent for a certain period of time followed by separation of the material from the water-miscible organic solvent. A decanter or a press is preferably used for this separation.

In the washing with the water-miscible organic solvent, the dry mass in the washing solution is from between 0.5 wt % and 15 wt %, preferably between 1.0 wt % and 10 wt %, and particularly preferably between 1.5 wt % and 5.0 wt %.

In the washing with the water-miscible organic solvent is preferably carried out with mechanical agitation of the washing mixture. Preferably, the washing is carried out in a vessel with a stirrer.

In the washing with the water-miscible organic solvent, advantageously a device for homogenization of the suspension is used. This device is preferably a toothed ring disperser.

According to an advantageous embodiment, washing with the water-miscible organic solvent takes place in a counterflow procedure.

In one embodiment, partial neutralization by addition of Na or K salts, NaOH or KOH, takes place during washing with the water-miscible organic solvent.

During washing with the water-miscible organic solvent, decoloring of the material can also be performed. This decoloring may take place by the addition of one or more oxidants. For instance, the oxidants could be chlorine dioxide and hydrogen peroxide, which may be used by themselves or in combination.

In an advantageous embodiment, during the at least two-fold washing with the water-miscible organic solvent, the concentration of the organic solvent in the solution increases with each washing step. By this incremental increase in organic water-miscible solvent, the portion of water in the fiber material is reduced in a controlled manner such that the rheological properties of the fibers are maintained during the subsequent steps of solvent removal and drying, and the activated fiber structure does not collapse.

Preferably, the final concentration of the water-miscible organic solvent amounts to between 60 and 70 vol % in the first washing step, between 70 and 85 vol % in the second washing step, and in an optional third washing step, between 80 and 90 vol %.

According to the optional step (g), the solvent content can additionally be reduced by contacting the material with water vapor. This is preferably done by means of a stripper in which the material is contacted with water vapor as the stripping gas in countercurrent.

In an advantageous embodiment, the material is moisturized with water after step (f) or (g) before drying. This is preferably done by introduction of the material in a moisturization screw and spraying with water.

In step (h), the washed material from step (f) or the stripped material from step (g) are dried, wherein the drying process comprises drying at normal pressure or vacuum drying.

Examples of suitable drying methods are fluid bed drying, fluidized-bed drying, belt drying, drum drying or paddle drying. Fluidized-bed drying is particularly preferred. The advantage is that the product is dried in a loose condition which simplifies the subsequent comminution step. In addition, this type of drying avoids damage to the product by local overheating since the input of heat can be dosed very well.

Drying under normal pressure in step (i) expediently takes place at a temperature between 50° C. and 130° C., preferably between 60° C. and 120° C. and particularly preferably between 70° C. and 110° C. After drying, the product is advantageously cooled to ambient temperature.

In an alternative embodiment the drying process according to step (h) comprises vacuum drying and preferably consists of vacuum drying. During vacuum drying, the washed material as drying material is subjected to a vacuum, which lowers the boiling point, thus leading to evaporation of the water even at low temperatures. The evaporation heat, which is continuously lost by the drying material, is suitably restored from outside up to constant temperature. The effect of vacuum drying is that it lowers the equilibrium vapor pressure, promoting capillary transport. This has in particular been proven advantageous for the present apple fiber material since in this manner the activated open fiber structures and the resulting rheological properties are maintained. Preferably, vacuum drying takes place at a negative pressure of less than 400 mbar, preferably less than 300 mbar, further preferably less than 250 mbar and particularly preferably less than 200 mbar.

Drying under vacuum in step (h) advantageously takes place at a shell temperature of between 40° C. and 100° C., preferably between 50° C. and 90° C. and particularly preferably between 60° C. and 80° C. After drying, the product is advantageously cooled to ambient temperature.

In an advantageous embodiment, the method additionally comprises a comminution, milling or sieving step after drying in step (h). This step is advantageously performed such that as a result, 90% of the particles have a size of less than 450 μm, preferably less than 350 μm and in particular less than 250 μm. At this particle size, the fiber is well dispersible and has optimum swelling properties.

The activatable, de-esterified fruit fiber used according to the invention, as well as a method to the production thereof, are disclosed in the application DE 10 2020 120 605.4.

In one embodiment, the activatable, de-esterified fruit fiber can be used for manufacturing of a food product. This can be any food product known to the person skilled in the art. Advantageously, the food product is selected from the group consisting of preserved products, deep-frozen foods, vegan food, vegetarian food, gluten-free food, low-calorie food, low-sugar food, lactose-free food, jelly, jelly-type sweets, sauce, granola bars, fruit pieces, fruit snacks, fruit bars, milk substitute drink, milk substitute product, foam sugar products, sherbet, ice cream, desserts, fermented drink, milk product, delicacies, fruit drink, fruit drink containing alcohol, cocktail, vegetable drink, chutney, barbecue sauce, smoothies, instant drink, fruit spread, fruit compote, fruit dessert, fruit sauce, fruit preparations, bake-stable fruit preparations, fruit preparations for yoghurt, bake-stable vegetable preparations, bake-stable fatty fillings, baked goods, pasta and pasta fillings, noodle dishes, potato snack, cheese and cream cheese preparations, meat substitute products, extruder products, corn flakes, breakfast cereals, soup, sauce, mayonnaise, meats, sausages, sausage casings, seafood, spirits, lozenges, functional food, nutritional supplements and dietary foods such as enteral foods, dysphagia food or sip feed.

In the food area, the activatable, de-esterified fruit fiber is especially suited for textured products. For further optimization, it can be combined here with hydrocolloids and/or functional dietary fibers.

In milk substitution drinks, such as, for example, almond milk, it has been shown that the activatable, de-esterified fruit fiber can increase stability and in particular contribute to turbidity stabilization. Additionally, the activatable, de-esterified fruit fiber can here increase viscosity, act as a good emulsifier and help to improve flavor release.

The use of the activatable, de-esterified fruit fiber in milk substitution products and milk products can provide the following advantages: increased stability, turbidity stabilization, better emulsification, better mouthfeel, texturing, reduction of nutritional value, increased creaminess, substitution of emulsifying salts, reduction of syneresis, improved spreadability and fat substitution.

Selected milk substitution products or milk products are, for example, desserts, yoghurt, yoghurt drink, non-fermented products, fermented drinks, fermented products, processed cheese, cream cheese products.

The use of the activatable, de-esterified fruit fiber in ice cream or frozen desserts can provide the following advantages: retardation of crystal growth, form stability in case of heating, improvement of melting behavior, fat substitution, increased creaminess, better mouthfeel, optimization of nutritional value, improved flavor release.

The ice cream or frozen dessert can here contain alcohol or not, be fat-free or high in fat content; it may contain insect protein, milk or milk components or even, as vegan ice cream, be free of animal protein. The ice cream or frozen dessert may also be fruit- and/or vegetable-based.

The use of the activatable, de-esterified fruit fiber in sweets and especially in chewing gum articles can provide the following advantages: improved abrasive behavior, water retention and improved flavor release.

The use of the activatable, de-esterified fruit fiber in sweets and especially in chocolate products can provide the following advantages: fat substitution, processing aid, process stability, better emulsification and thus reduction of grease leak, better viscosity, texturing, optimization of nutritional value (e. g. by reduction in sugar).

The use of the activatable, de-esterified fruit fiber in sweets and especially in jelly-type products can provide the following advantages: texturing, improved gelling, adaptation of viscosity, process optimization, reduction in stickiness and better processing.

Some examples of respective sweets are: fruit pieces, jelly products with different Brix values, jelly products containing fruit, jelly products containing vegetables, these jelly products in combination with nuts or nut derivatives, and sweet confectionary fillings.

The use of the activatable, de-esterified fruit fiber in fruit- and/or vegetable-containing drinks potentially containing additional products such as cereals, nuts etc. can provide the following advantages: increased stability, turbidity stability, good emulsification of juices, better mouthfeel, texturing and reduction in nutritional value.

The fruit- and/or vegetable-containing drink can comprise a large range in terms of viscosity, ranging from thin-flowing to spoonable. In addition to sugary drinks, drinks which are reduced in sugar, sugar-free or salty can also be used. Smoothies are preferred.

The use of the activatable, de-esterified fruit fiber in bake-stable fillings can provide the following advantages: form stability, reduction in syneresis, easy introduction, enhanced processing. Advantageously, the activatable, de-esterified fruit fiber can be employed for fillings with a low Brix value of 30-45% dry matter or even lower.

The bake-stable fillings can be fillings containing fruit, vegetables, chocolate, nuts, cereals, cheese or any combination thereof.

The use of the activatable, de-esterified fruit fiber in deep-frozen products, and in particular deep-frozen bakery products, can provide the following advantages: enhanced stability of deep-frozen bakery products in terms of loss of volume over storage time, network stabilization, support of gelling in the bakery product and support of gluten network stability.

The use of the activatable, de-esterified fruit fiber in baked goods can provide the following advantages: improved elasticity of dough, prolonged freshness, delayed retrogradation, reduction of surface stickiness, improved machine runability (e. g. in case of rye and spelt), optimization of break stability, maintaining of crispness, enhancement of yield of dough and reduction of pastry loss.

The use of the activatable, de-esterified fruit fiber in sprinkled baked goods can provide optimized adhesion to e. g. cereals, spices or the like. This applies to frozen and non-frozen products.

The use of the activatable, de-esterified fruit fiber in gluten-free baked goods can provide the following advantages: improved elasticity of dough, prolonged freshness, delayed retrogradation, reduction of surface stickiness, improved machine runability, optimization of break stability, maintaining of crispness, enhancement of yield of dough, reduction of pastry loss. The activatable, de-esterified fruit fiber here provides a substantial contribution to viscosity build-up. It also supports the starch network.

The use of the activatable, de-esterified fruit fiber in extrudates can provide the following advantages: support of extrudability, improved volume result, fine pore structure. This applies to a broad spectrum of extruded products, such as e. g. cereal, fruit, vegetable, protein or meat extrudates.

The use of the activatable, de-esterified fruit fiber in meat substitutes based on plant proteins can provide the following advantages: enhanced form stability, enhanced water retention, enhanced emulsification, advantageous texturing, bite optimization, stabilization of matrix, improved cohesion.

The use of the activatable, de-esterified fruit fiber in savory products can provide the following advantages: reduction of syneresis, advantageous texturing, stabilization, easy introduction, good form stability, maintaining/support of the typical structure.

The use of the activatable, de-esterified fruit fiber in soups or sauces can provide the following advantages: spillover protection due to gelation at respective temperatures, fusion at respective temperatures, optimum gelling; better mouthfeel, good emulsification, stabilization, advantageous texturing.

The use of the activatable, de-esterified fruit fiber in products based on insects or insect proteins can provide the following advantages: better form stability, enhanced water retention, enhanced emulsification, advantageous texturing, bite optimization, stabilization of matrix, improved cohesion.

The use of the activatable, de-esterified fruit fiber in meat and sausage products can provide the following advantages: reduction or substitution of added salts (e.g. phosphates), increased water binding, enhanced emulsification, optimization of cutting properties, enhancement of elasticity, increased water retention, delayed drying on the surface, fat substitution, optimization of nutritional value (e. g. by fat or salt reduction).

The use of the activatable, de-esterified fruit fiber in products containing alcohol can provide the following advantages: stabilization at alcohol contents to be defined, good viscosity adjustment, enhanced emulsification, good water binding, better mouthfeel and increased creaminess. These products can comprise a broad spectrum, from spirits such as liqueurs and jellies containing alcohol down to fillings containing alcohol.

The use of the activatable, de-esterified fruit fiber in instant products can provide the following advantages: good carrier substance or good separating agent between the functional components, good viscosity build-up in cold or hot media, enhanced emulsification, advantageous texturing, stabilization and good dispersibility.

The use of the activatable, de-esterified fruit fiber in artificial, i. e. in particular plant-based casings, can provide the following advantages: softer casing, optimized elasticity, good coating of the casings. Here, a combination with pectin is advantageous.

The use of the activatable, de-esterified fruit fiber in dietary foods and in particular enteral feeding can provide the following advantages: good viscosity and ductility, easy swallowing of the food, homogeneous distribution of the agents contained.

The use of the activatable, de-esterified fruit fiber in nutritional supplements can provide the following advantages: good viscosity, increase in dietary fiber content, stabilization, advantageous mouthfeel, fat substitute, good texturing, good emulsification.

The activatable, de-esterified fruit fiber used according to the invention can be used as a foaming agent or whipping agent for foam stabilization. The possible advantages are: increased stability, enhanced formation and stability of emulsions, better mouthfeel, texturing, reduction in nutritional value, increased creaminess, enhanced spreadability, fat substitute, optimized destabilization of fat agglomerates.

Selected products for this type of use are foamed desserts (milk- or non-milk-based), cream, Froop® (creamy yoghurt with top layer of fruit puree) and ice cream.

The activatable, de-esterified fruit fiber used according to the invention can be employed as an emulsifier. The possible advantages include: improved gloss, better mouthfeel, fat substitute, increased creaminess, no overemulsification, better formation and stability of emulsions, optimization of nutritional value, texturing, stabilization and optimization of yield point. The activatable, de-esterified fruit fiber can here be used for emulsions with a great variety of fat contents: from fat-free emulsions up to 80% of fat content.

The activatable, de-esterified fruit fiber used according to the invention can be used as a carrier substance. For instance, it can be a carrier of active agents, flavors or colors.

The activatable, de-esterified fruit fiber used according to the invention can be employed as a separating agent or free flow enhancer. It forms a protective layer between hygroscopic surfaces. Here, ease of use is advantageous.

The activatable, de-esterified fruit fiber used according to the invention can be employed for manufacturing textile fibers and thus, textiles.

In one embodiment, the activatable, de-esterified fruit fiber can be used for producing feedstuff. The person skilled in the art can employ all types of feedstuff known to him as products. Advantageously, the feedstuff is selected from the group consisting of feedstuff rich in starches, oleaginous feedstuff, feedstuff rich in protein, extrudate feedstuff, wet feed, binder, bird feedstuff rod, rodent feedstuff rod, fish bait, supplement feedstuff, feedstuff for particular nutritional purposes and dietary feedstuff.

The use of the activatable, de-esterified fruit fiber in feedstuff in the form of wet feed can provide the following advantages: good texturing and structuring, good emulsification, stabilization, enhanced flavor release and optimization of nutritional value.

The use of the activatable, de-esterified fruit fiber in feedstuff in the form of extrudates can provide the following advantages: finer pore structure and better volume result.

In one embodiment, the activatable, de-esterified fruit fiber can be used for the production of animal supplies. The person skilled in the art can employ all types of animal supplies known to him as products. Advantageously, the animal supply is an animal bedding.

The use of the activatable, de-esterified fruit fiber in animal bedding can provide the following advantages: high water absorption capacity and good retention.

In one embodiment, the activatable, de-esterified fruit fiber can be employed for producing a hygiene article. Here, the person skilled in the art can use all hygiene articles known to him as products. Advantageously, the hygiene article is selected from the group consisting of wet wipes, diapers, incontinence articles such as protective trousers or incontinence pants, sanitary towels, tampons, panty liners and softcups.

The use of the activatable, de-esterified fruit fiber in products such as wet wipes can result in good water binding and good water retention capability.

In one embodiment, the activatable, de-esterified fruit fiber can be employed for producing a personal care product. The person skilled in the art can use all personal care products known to him as products. Advantageously, the personal care product is selected from the group consisting of soap, shower gel, bath additives, skin creams, lotions, gel, sunscreen, sun cream, repellent, shaving cream, shaving soap, epilator cream, toothpaste, dentition adhesive medium, shampoo, hair forming agents, hair-setting products, hair colorants, facial make-up, eye care products, lip care products, nail polish and self-tanning agents.

The use of the activatable, de-esterified fruit fiber in products such as toothpaste, dentition adhesive medium or casting compounds can provide the following advantages: good abrasiveness, good adhesion, smooth and soft mouthfeel, good emulsification, targeted viscosity formation, stabilization, control of gelling speed.

The use of the activatable, de-esterified fruit fiber in products such as shampoos or creams can result in vitalization, moisture-stabilizing effect on the skin (delayed drying out) and good skin tolerability.

The use of the activatable, de-esterified fruit fiber in liquid-absorbing products such as diapers, incontinence articles like protective trousers or incontinence pants, sanitary towels, tampons, panty liners and softcups, can provide the following advantages: high water absorption capability and good retention.

In one embodiment, the activatable, de-esterified fruit fiber can be used for producing a cleaning agent. Here, the person skilled in the art can use all cleaning agents known to him as products.

Advantageously, the cleaning agent is selected from the group consisting of detergent, bile soap, washing-up liquid, dishwasher detergent, rinsing agent, neutral cleaner, abrasive cleaner, window cleaner, lime remover, drain cleaner, brake cleaner, alcohol cleaner, all-purpose cleaner, glass cleaner, sanitary cleaner, toilet cleaner, toilet gel, toilet soap, carpet cleaner, car care material, oven cleaner, bathroom cleaner and metal polish, shoe polish, oil absorbing and anti-dust agent.

The use of the activatable, de-esterified fruit fiber in detergents can provide the following advantages: good adhesion to the toilet wall, good and stable gelling, advantageous abrasiveness, good solubility.

The use of the activatable, de-esterified fruit fiber in toilet gels or toilet soaps can provide the following advantages: as a separating agent, a good separation of the functional components and homogeneous distribution of the abrasive components and agents.

The use of the activatable, de-esterified fruit fiber in liquid detergents, and in particular in washing-up liquids, can provide the following advantages: as a separating agent, a good separation of the functional components and homogeneous distribution of the abrasive components and agents; good emulsification.

The use of the activatable, de-esterified fruit fiber in shoe polish can provide the following advantages: good and stable emulsification, advantageous texturing.

In one embodiment, the activatable, de-esterified fruit fiber can be employed for producing a coating agent. The person skilled in the art can use all coating agents known to him as products. Advantageously, the coating agent is selected from the group consisting of antistatic coating, oil-repellent coating and anti-block coating.

In one embodiment, the activatable, de-esterified fruit fiber can be employed for producing an explosive. The person skilled in the art can use all explosives known to him as products. Advantageously, the explosive is a gelatinous explosive.

The activatable, de-esterified fruit fiber can be employed as a separating agent in the explosive. It can reduce hygroscopicity, control gelation and facilitate processing.

In one embodiment, the activatable, de-esterified fruit fiber can be employed for producing a lubricant. The person skilled in the art can use all lubricants known to him as products. Advantageously, the lubricant is selected from the group consisting of liquid lubricants, such as lubricating oil and cooling lubricant, lubricating grease and solid lubricant.

The use of the activatable, de-esterified fruit fiber in a lubricant can provide the following advantages: targeted control of viscosity and yield point, stabilization of the emulsion.

The use of the activatable, de-esterified fruit fiber in a coolant can provide the following advantages: targeted control of viscosity and yield point, and thus optimized energy absorption for improving cooling capacity.

In one embodiment, the activatable, de-esterified fruit fiber can be employed for producing a plastic product. The person skilled in the art can use all plastic products known to him as products. Advantageously, the plastic product is a citrus fiber-reinforced plastic or a wood-plastic composite (WPC).

Production of an alternative plastic product is advantageously performed by production of a compacted product. In this manner, flowerpots, straws or pallets, for example, can be produced.

In one embodiment, the activatable, de-esterified fruit fiber can be employed for production of a varnish. The person skilled in the art can use all varnishes known to him as products. Advantageously, the varnish is selected from the group consisting of alkyd resin varnish, oil varnish, cellulose nitrate varnish, bitumen varnish, tar-containing varnish, phenolic resin varnish, urea resin varnish, melamine resin varnish, polyester varnish, epoxy resin varnish, polyurethane resin varnish, acrylic varnish and powder varnish.

In one embodiment, the activatable, de-esterified fruit fiber can be employed for production of a coating agent. Here, the person skilled in the art can use all coating agents known to him as products. Advantageously, the coating agent is selected from the group consisting of glaze, oil paint, dispersion paint, chalk paint, silicate paint and liquid plaster.

The use of the activatable, de-esterified fruit fiber in coating agents can provide the following advantages: targeted adjustment of viscosity, good emulsion stabilization and adjustment of yield point, better material adhesion, enhanced processability, e. g. in terms of spreadability or sprayability.

In one embodiment, the activatable, de-esterified fruit fiber can be employed for production of a construction material. The person skilled in the art can use all construction materials known to him as products. Advantageously, the construction material is selected from the group consisting of building foam, sound proofing material, insulation material, concrete, screed, mortar, cement, chemical bonded anchors, chemical anchor bolts, asphalt and whisper asphalt.

The addition of the activatable, de-esterified fruit fiber to an asphalt mixture results in the formation of a low-noise “whisper asphalt”.

The addition of the activatable, de-esterified fruit fiber to construction materials such as concrete, screed, mortar or cement of an asphalt mixture can lead to controlled drying, reduction in the formation of cracks, optimized long-time durability and control of hardening.

The addition of the activatable, de-esterified fruit fiber to a sound proofing material or an insulation material can stabilize the matrix and reduce the transmission of heat and sound.

In case of a building foam, the activatable, de-esterified fruit fiber can stabilize the foam, thus advantageously influencing the matrix structure.

In one embodiment, the activatable, de-esterified fruit fiber can be employed for producing an adhesive. The person skilled in the art can use all adhesives known to him as products. Advantageously, the adhesive is selected from the group consisting of dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive and paste.

In adhesives, and in particular in paste, the activatable, de-esterified fruit fiber can help to control viscosity in a targeted manner and to improve spreadability.

In one embodiment, the activatable, de-esterified fruit fiber can be used for production of a medical product. The person skilled in the art can use all medical products known to him as products. Advantageously, the medical product is selected from the group consisting of powder, juice, lotion, ointment, cream, gel, tablet and rubber article.

The use of the activatable, de-esterified fruit fiber in ointments can provide the following advantages: good control of viscosity, good formability, easy swallowing, increased creaminess, homogeneous distribution of agents, good drying, increased stabilization, good emulsification and good skin compatibility.

In one embodiment, the activatable, de-esterified fruit fiber can be used for production of a medical product. The person skilled in the art can use all known medical products as products. Advantageously, the medical product is selected from the group consisting of wound dressing, emergency bandage, transdermal patch, stoma product and dental casting compound.

The use of the activatable, de-esterified fruit fiber in patches can provide the following advantages: good gelling and water absorption with retention of the absorbed liquid. This results in moisture-stabilizing patches.

The use of the activatable, de-esterified fruit fiber in stoma products, such as colostomy bags, can provide the following advantages: good water absorption and water binding with retention of the absorbed liquid, good skin compatibility.

In one embodiment, the activatable, de-esterified fruit fiber can be used for production of a battery. The person skilled in the art can use all batteries known to him as products. Advantageously, the battery is selected from the group consisting of primary cell, accumulator and solid cell battery.

In one embodiment, the activatable, de-esterified fruit fiber can be employed in the construction area. Advantageously, the use in road and path construction, masonry construction, concrete construction and reinforced concrete construction is comprised.

In one embodiment, the activatable, de-esterified fruit fiber can be employed in extraction by drilling boreholes. Usage as addition to drilling fluid or fracfluid is advantageous.

The use of the activatable, de-esterified fruit fiber in a drilling fluid or a fracfluid can provide the following advantages: increased viscosity in “drilling mud” or similar drilling liquids, replacement of the oil by medium with higher viscosity, targeted adjustment of viscosity, oil binding, good emulsification. As a result, the activatable, de-esterified fruit fiber can thus be used as an extraction aid in the mining industry.

In one embodiment, the activatable, de-esterified fruit fiber can be employed in agriculture. The usage in fertilizers, humectants, soil conditioners, plant substrates, flowerpots or substrate-tablet extrudates is advantageous.

In one embodiment, the activatable, de-esterified fruit fiber can be employed for producing a fertilizer. The person skilled in the art can use all fertilizers known to him as products. Advantageously, the fertilizer is a binder for fertilizer cones.

The use of the activatable, de-esterified fruit fiber in the production of fertilizers can help to keep the agents suspended and to control viscosity and yield point in a targeted manner.

In a substrate-tablet extrudate, the activatable, de-esterified fruit fiber can serve as a carrier and/or a separating agent. The pectin can be detached from the fibers and release the nutrients in an ordered manner. In can also support moisturization.

In one embodiment, the activatable, de-esterified fruit fiber can be employed as a reinforcing agent for producing a composite material. The person skilled in the art can use all composite materials known to him as products. Advantageously, the activatable, de-esterified fruit fiber is employed for targeted control of abrasive properties, here as a substitute for microplastics.

As an alternative, the activatable, de-esterified fruit fiber can be used for surface treatment of the composite materials.

The use of the activatable, de-esterified fruit fiber in the production of a composite material can optimize durability and lead to improved elasticity.

In the previously taught uses, the activatable, de-esterified fruit fiber is preferably a de-esterified citrus fiber or a de-esterified apple fiber.

In a further aspect, the invention relates to a product selected from the group consisting of food products, feeding stuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, wherein the product comprises the activatable, de-esterified fruit fiber. Here, the activatable, de-esterified fruit fiber is preferably a de-esterified citrus fiber or a de-esterified apple fiber.

In one embodiment, the product contains a portion of 0.05 wt % and 90 wt %, preferably between 0.1 and 50 wt %, particularly preferably from 0.1 to 25 wt % and especially preferably between 0.5 and 10 wt %, of the activatable, de-esterified fruit fiber. For instance, the portion of the activatable, de-esterified fruit fiber may be 1.0%, 1.25%, 1.5%, 1,75%, 2.0%, 2.25%, 2.5%, 2.75%, 3.0%, 3.25%, 3.5%, 3.75%, 4.0%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88% or 89%, wherein these are percentages by weight. Here, the activatable, de-esterified fruit fiber is preferably a de-esterified citrus fiber or a de-esterified apple fiber.

Definitions

A fruit fiber according to the application is a plant fiber, i.e. a fiber, which is isolated from a non-lignified plant cellular wall and consists mainly of cellulose, and which is here isolated from a fruit. A fruit in this context is understood to mean the entirety of the organs of a plant that arise from a flower, and includes both the classical fruit fruits and fruit vegetables.

An “apple fiber” in the sense of the application is a component consisting mainly of fibers, which is isolated from a non-lignified cellular wall of an apple and consists mainly of cellulose. In a sense, the term “fiber” is a misnomer since macroscopically, the apple fibers do not appear as fibers but as a powdery product. Other components of the apple fiber are, among others, hemicellulose and pectin.

The apple fiber can be obtained from all cultivated apples (malus domesticus) known to the person skilled in the art. As starting material, advantageously processing residues of apples can be employed. The starting material may be apple peel, apple core, apple seeds, fruit flesh or a combination thereof. Preferably, apple pomace is used as the raw material, i. e. the press residues of apples, which typically also contain the above-mentioned components in addition to the peels.

A “citrus fiber” in the sense of the application is a component consisting mainly of fibers, which is isolated from a non-lignified cellular wall of a citrus fruit and consists mainly of cellulose. In a sense, the term “fiber” is a misnomer since macroscopically, the citrus fibers do not appear as fibers but as a powdery product. Other components of the citrus fiber are, among others, hemicellulose and pectin. The citrus fiber can advantageously be obtained from citrus pulp, citrus peel, citrus vesicle, segment membranes or a combination thereof.

Within the context of the invention, a “fatty cream” is understood to be a cream containing cooking oil and/or cooking fat. Cooking fats and cooking oil are lipids suitable for human consumption and having a neutral or characteristic smell and flavor. The substances are either called “cooking fat” or “cooking oil”, depending on whether they are solid or liquid at room temperature.

An activatable, de-esterified fruit fiber according to the present application is defined by a content of water-soluble pectin of between 2 to 10 wt %, wherein this pectin is a low methoxyl pectin.

An activatable, de-esterified apple fiber according to the present application is defined by a content of water-soluble pectin of between 2 to 10 wt %, wherein this pectin is a low methoxyl pectin.

An activatable, de-esterified citrus fiber according to the present application is defined by a content of water-soluble pectin of between 2 to 10 wt %, wherein this pectin is a low methoxyl pectin.

The expression “bake-stable” according to the invention is used to indicate the fact that if subjected to dry heat, a fat-containing creamy composition only expands to a minimum (i. e. by maximally 25%), as can be determined by the following baking test method. For this purpose, a preparation is used which before the test, in the cooled state, has a creamy and paste-like consistency, such as a chocolate cream, a fruit preparation or a vegetable preparation. A metal ring of 1 cm height and 60 mm in diameter is placed on a piece of filter paper (Hahnenmühle company, Dassel Germany, type 589/1, DP 5891 090, 0 90 mm) and is filled with the preparation to be tested, which is placed on the filter paper and smoothed out on the surface of the metal ring. After evenly drawing off the metal ring, the filter paper, on which the preparation has been spread, is placed on a baking tray and baked in the preheated oven (top and bottom heat) for 10 minutes at 200° C. The form stability (diameter before baking in relation to the diameter after baking) of the preparation is assessed. The diameter of the preparation after baking may at most be 125% of the diameter before baking.

A soluble pectin according to the application is defined as a plant polysaccharide which, as a polyuronide, substantially consists of α-1,4-glycosidically bonded D-galacturonic acid units. The galacturonic acid units are partially esterified with methanol. The degree of esterification describes the percentage degree of carboxylic groups in the galacturonic acid units of the pectin which are present in esterified form, e. g. as methyl esters.

A high methoxyl pectin according to the invention is a pectin with a degree of esterification of at least 50%. A low methoxyl pectin, in contrast, has a degree of esterification of less than 50%. The degree of esterification describes the percentage degree of carboxylic groups in the galacturonic acid units of the pectin which are present in esterified form, e. g. as methyl esters. The degree of esterification can be determined by the method according to JECFA (monograph 19-2016, Joint FAO/WHO Expert Committee on Food Additives).

An “instant product” in the sense of the present invention is defined to be a semi-finished food product generally consisting of powder, granulate or dried ingredients, which is stirred into a cold or hot liquid. Cooking during preparation is omitted.

The term “seafood” in the present application is intended to indicate all edible marine animals which are no vertebrates. Typical seafood are clams and aquatic snails, squids and octopi, shrimps, crabs, crayfish and lobster. Seafood can be caught or be a farmed product.

An “extruder product” (synonymous with extrusion product) according to the invention is a product produced by extrusion, which is in general crispy and/or expanded and which can assume any desired shape depending on the nozzles used in the extrusion process. Examples of extruder products are: snacks such as peanut flakes, breakfast cereals, flatbreads, pasta products, sweets such as marshmallows and various extruded soy products which are used in numerous industrially produced foods, both as individual products and as components.

A “smoothie” is a cold mixed drink consisting of fruit and optional milk products, which is freshly prepared or sold as a finished product. Other than in fruit juices, the entire fruit and partly also the peel are used in smoothies. The basis of smoothies is therefore the fruit pulp or fruit puree which, depending on the recipe, is mixed with juices, water, milk, milk products or coconut milk so as to obtain a creamy and smooth consistency.

Within the context of the invention, a “nutritional supplement” is defined to be a food whose purpose it is to supplement the general diet; which is a concentrate of nutrients or other substances with a dietary or physiological effect, by itself or in combination; and which is marketed in dosed form, especially as capsules, lozenges, tablets, pills, effervescent or other similar forms of administration, powder bags, drop-dispensing bottles, bottles with dropper insert and other similar administration forms of liquids and powders for dosage in small metered amounts.

A “functional food” within the context of the invention is characterized by not only having a nutritional value and providing flavor experience, but in addition serving the purpose of long-term promotion and maintenance of health as a “functional” component. Thus, functional foods are predominantly used for preventive healthcare, improving health status and well-being. Important targets of functional foods are the gastrointestinal tract, the cardiovascular system, skin and the brain. Functional foods are consumed in a normal manner and not formulated as tablets, capsules or powders, other than nutritional supplements. The biologically active ingredients of functional foods are called nutraceuticals, which term is to designate their health-promoting effects. Frequently, probiotics and prebiotics, phytochemicals, omega-3 fatty acids, vitamins and dietary fibers are added to functional foods as nutraceuticals.

In the context of the invention and in accordance with the Deutsche Diatverordnung (German dietary regulation), a “dietary food” is defined to be a food destined for a specific group of people and for specific nutritional purpose; in addition, it significantly differs from food for general consumption. They are not used for the general diet of average consumers but for a well-defined group of people, such as people with disorders of digestion, resorption and metabolism, people in “particular physiological conditions” or healthy babies and infants.

The following groups of foods are examples of dietary foods: infant formulae and follow-on formulae; other foods for babies and infants (supplementary diet); low-calorie foods for weight loss; foods for special medical purposes (balanced diets); low-sodium foods including dietary salts which have a low sodium content or are entirely free of sodium; gluten-free foods (without additives); foods for intense muscle training, above all, for athletes; food for people suffering from disorders of the glucose metabolism (diabetics), enteral feeding and liquid food.

“Enteral food” according to the invention is a food which is liquid and of a viscosity low enough so that it can be administered via a feeding tube. It is a completely balanced diet for enteral feeding which is applied via a tube and an application system by the force of gravity or via a pump system. The standard foods cover all requirements of humans for carbohydrates, fats, protein, vitamins and trace elements, additionally containing dietary fibers. An isocaloric standard food has approximately 1.0 to 1.2 kcal/ml with a water content of 80% to 85%. If the energy density is higher, the diet involves high caloric standard food with a lower water content of 64% to 77%, which must be taken into account for liquid balancing.

“Liquid food” within the context of the present invention is a specially composed high-energy food in liquid form which can be drunk. It is used for additional or complete feeding if the patient cannot consume solid foods or can do it only to an insufficient degree.

“Feedstuff” (also briefly called feed) according to the invention is a collective term for all types of animal feed. The term comprises the food for all animals kept by humans, such as livestock, zoo animals, animals for sports or pets. Feedstuff today is specifically formulated for the respective species and purpose. Some examples are: feedstuff rich in starches which is produced from grains, seeds and tubers rich in starches; oleaginous feedstuff, feedstuff rich in protein, that is, containing 35-65% of protein, and other feedstuff obtained either from nature (e. g. fishmeal) or as an afterproduct from industrial production. These are, for instance, bran (from the mill), stillage (production of alcohol), brewer grains (production of beer), pomace (production of wine and juice), molasses and beet pulp from the sugar industry, and other food residues.

“Animal bedding” according to the invention designates materials which are used in animal husbandry for covering the ground in stables and cages and absorb the animals' excrements.

A “wound dressing” is a dressing placed on external wounds in order to prevent the entry of foreign bodies into the wound and absorb blood and wound exudate. In addition, wound dressings may guarantee a warm and humid wound climate promoting healing, alleviate pain by means of contained substances, promote wound healing or have antimicrobial effects.

A “commodity good” in the sense of the present application is an article which, in accordance with §2 subparagraph 6 of the German Lebensmittel-, Bedarfsgegenstände- and Futtermittelgesetzbuch (LFGB; Code on food, commodity goods and feedstuff), is selected from the list consisting of:

no. 1 materials and objects designed to come in contact with food (also called food commodity goods),

no. 2 packaging, containers or other casings designed to come in contact with cosmetics,

no.3 objects designed to come in contact with the mucosae of the mouth,

no. 4 objects designed for hygiene,

no. 5 toys and joke articles,

no. 6 objects designed to come not only temporarily in contact with the human body, such as clothing, bed cloth, masks, wigs, hairpieces, artificial eyelashes, wristbands,

no. 7 cleaning and care articles designed for domestic use or for commodity goods in the sense of number 1,

no. 8 impregnation agents and other equipment for commodity goods in the sense of number 6 which are designed for domestic use,

no. 9 means and objects for the improvement of smell in rooms designed for the accommodation of people.

A “filtering aid” according to the invention is a chemically inert substance which physically-mechanically supports filtration. It is not supposed to be confused with or equated to a flocculant. Filtering aids are employed to facilitate cleaning of the actual filter or filter insert or to prevent solid substances in the suspension from clogging the filter or getting into the filtrate. Filtering aids are generally used in water treatment, filtration of drinks and more specifically in the chemical industry.

An “egg substitute” according to the invention is a plant-based food which optically or in terms of taste as well as in its properties in the preparation of dishes resembles the whole egg, the egg white or the yolk. Use of a plant-based egg substitute can be associated with easier handling, a lower price and a lesser risk of food poisoning.

A “glazing agent” according to the invention is a food additive which protects the food from losses in smell, taste and moisture, promotes gloss or prolongs freshness. It can also function as a separating agent.

A “humectant” according to the invention is a food additive which prevents the drying out of foods by binding water added during production (i. e. preventing evaporation) or by attracting humidity of the air during storage. By preventing hardening of the finished food, it acts as a softening agent. In sweets, it counteracts crystallization of the sugar.

A “dietary fiber” according to the invention is a largely indigestible food component, generally consisting of carbohydrates, which predominantly exist in plant-based foods. For purposes of simplicity, dietary fibers are classified into water-soluble fibers (like pectin) and water-insoluble fibers (such as cellulose). Dietary fibers are considered an important part of the human diet. The EU Regulation on nutrition labelling globally assigns them a calorific value of 8 kJ/g.

A “reinforcing agent” according to the present invention is a single substance of a composite material. As the name suggests, the reinforcing agent is to guarantee rigidity and stiffness of the composite. What is most significant, in addition to its type, is the form of the reinforcing agent, namely whether it is present as a particle, a fiber or in layers. In particular, by “reinforcing agents” (“reinforcement”) the organic additives employed in plastics are understood which reinforce the plastics matrix. By “reinforcement”, the enhancement of mechanical and physical properties, such as elasticity, bending strength, creep mechanisms and heat deflection temperature are understood. Reinforcing agents are employed in a targeted manner to improve these material properties.

“Gelling agents” according to the invention are food additives which swell in water or bind water, i. e. lead to gelling. They form a gelatinous mass and give a viscid or solid consistency to soups, sauces or pudding.

A “solidifier” according to the invention is a food additive which ensures that solidity and freshness of a food remain during and after processing. It reacts with various components, such as e. g. pectin. A solidifier can be, for instance, a calcium salt which reacts with an ingredient of the product, such as the pectin in fruit.

A “texturant” according to the present application is a substance with the ability to provide a special texture to a product. By “texture”, in this context, the surface properties of food are understood which can be perceived by the tactile sense, in particular the mouthfeel of a product.

A “thickener” according to the present application is a substance which is, above all, capable of binding water. By removing unbonded water, viscosity is increased. Above a certain concentration, which is characteristic for each individual thickener, network effects are additionally produced which lead to a generally disproportionate increase in viscosity. Thickeners are capable of providing a product with a specific consistency. Thickening in this context means an increased viscosity or solidity of the product as a result of employment of the thickener.

A “filler” according to the invention is an insoluble additive which, added in large amounts to the basic material (of the matrix), strongly alters, among others, the mechanical, electric and processing properties of materials and can at the same time significantly decrease the portion of the matrix, which is typically more expensive, in the finished product. Preferably, a filler is a food additive which forms part of the food volume without substantially contributing to its content of usable energy. In this manner, the actual energy content per volume or per mass of the foods is reduced.

A “carrier” according to the invention is a substance to which other substances can be attached (physically bonded), i. e. which can “carry” other substances. For instance, a pharmaceutical agent or an aroma ingredient, which is otherwise difficult to dose, can be bonded to a carrier which is easier to dose. Preferably, the carrier is a technical adjuvant in the food industry and can transport flavors into the products, with the appearance and taste of a food generally not being modified by the carrier itself. As technical adjuvants, they do not have to be listed in the list of ingredients since they in themselves do not cause effects in the final product.

Within the context of the present invention, an “emulsifier” is an adjuvant which is used to combine two fluids which by themselves do not mix, such as oil and water, to form a finely distributed mixture, called an emulsion, and to stabilize it. The same applies to the mixing of solid, non-soluble substances with a liquid in order to stabilize a suspension. Preferably, the emulsifier is a food additive.

A “separating agent” according to the invention is a food additive or technical adjuvant which prevents sticking together or agglomeration of foods. Thus, separating agents are also among the agents which increase or maintain flowability. Thus, separating agents prevent salt, for example, from turning lumpy and individual pieces of candy from sticking together and forming one single block of sugar. As technical adjuvants, they are employed in industrial processing and production of foods. Technical adjuvants are food additives which are added in order to facilitate technical processes, such as cutting and filtration. In the final product, however, the technical adjuvants may not be present at all or only in low residual amounts which are unavoidable.

A “free flow agent” according to the invention is a separating agent which is added to crystalline substances in order to prevent agglomeration of the individual crystals, mainly for the purpose of better machine processability. Its use is to prevent the lumping of, for instance, table salt before or during processing, which makes it more difficult to dose.

A “stabilizer” according to the invention is a food additive which has the property of maintaining, if it is added to a metastable system, the characteristics, manageability, flavor or other parameters of this system in a defined manner, and thus of stabilizing it. A stabilizer can serve one or more functions.

An “improving agent for baking stability” according to the invention is characterized in that a liquid, viscous or cream-like composition, to which the improving agent is added, only spreads or flows minimally after the agent has been added and dry heat is applied.

A “foaming agent” according to the invention is a food additive which ensures that a homogeneous dispersion of gas forms in liquid or solid foods. Thus, foaming agents ensure that gases distribute evenly in liquids or solids.

A “whipping agent” according to the invention is an food additive which, after it has been added to a mass, allows increasing the volume of the mass by whipping air into it. Whipping agents stabilize the mass and thus facilitate the handling thereof. Whipping agents are used in the food industry for instance to produce sponge cake, chocolate mousse and other desserts.

An emergency bandage, also colloquially called an adhesive plaster or patch, is a piece of wound dressing attached to an adhesive tape. It is used to cover small wounds.

A “transdermal patch” according to the invention is a dosage form for the systemic administration of drugs in the form of patches. It is attached to the skin and releases the agent in a controlled manner, which agent is then resorbed by the skin. The agent reaches the circulatory system without previously having been broken down in the gastrointestinal tract or the liver.

Within the framework of the application, a “stoma” is understood to be an artificial connection between a body cavity and the body surface. Typical examples of a stoma are colostomy, ileostomy and urostomy. For the receiving of excrements, such as stool and/or urine, stoma products (e. g. ileostomy bags) are employed. These are bags attached to an adhesive surface. The adhesive surface is attached to the abdomen around the stoma and adheres to the skin.

Within the framework of the application, “cleaning agents” are consumables which are used to clean various articles and objects. The cause or support the removal of impurities due to usage, or of residues and adhering substances from the manufacturing process of the object. Different fields of application require different cleaning agents. For laundry and textiles, detergents (heavy-duty detergents, color detergents, fabric softeners etc.) or bile soap are employed. For dishes (cookware, dinnerware and cutlery) washing-up liquid, dishwashing agents or rinsing aids are used. For surfaces in living spaces and offices: neutral cleaners, abrasive cleaners (scouring powder) or window cleaners. Other cleaning agents are, for instance, lime removers, drain cleaners, brake cleaners, alcohol cleaners, all-purpose cleaners, glass cleaners, sanitary cleaners, toilet cleaners, carpet cleaners, car care material, oven cleaners, bathroom cleaners and metal polish.

In the context of the present application, a “lubricant” is a substance used for lubrication which serves to reduce friction and wear as well as to provide cooling, vibration damping, sealing and protection against corrosion. Principally, all lubricants consist of a basic liquid (generally base oil) and of other ingredients called additives. Examples of lubricants are liquid lubricants (lubricating oils and cooling lubricants), lubricating greases and solid lubricants (such as graphite).

Within the framework of the invention, “coolants” are liquid or solid substances or mixtures of substances which are used for the dissipation of heat.

A “composite material” is a material consisting of two or more bonded substances which has material properties different from those of its individual components. For the properties of composite materials, substantial properties and the geometry of the components are important. Size effects play a particular role.

Within the context of the present invention, “paints” are liquid or pasty, rarely powdery substances of mixtures which, if applied on surfaces, are subjected to physical drying or chemically curing processes. According to DIN 55945, a paint is a “liquid or pasty coating agent which is mainly applied by brushing or rolling”.

An “adhesive” according to the invention is a non-metal substance which is capable of bonding materials by surface (adhesion) and its inner stability (cohesion). That is, it is a process material employed to bond different materials in the adhesive bonding method. Examples are dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive.

“Drilling fluids” (also called drilling mud) in the context of the present application are liquids which are pumped through the borehole in mining. There are two basic types of drilling fluids; those based on water and those based on oil. Drilling fluids are basically used for stabilizing a borehole, cleaning the borehole bottom and removing the bottom material (cuttings) which have been drilled out. In addition, they dissipate the frictional heat which has been induced on the trepan, thus cooling and lubricating the drilling tool. In addition, they reduce the frictional resistance of the trepan and the rotating boring rods and dampen their oscillations.

Fracking is a method of creating, widening and stabilizing cracks in the rocks of a deep deposit with the aim of increasing the permeability of the deposit rocks. In this manner, gases or liquids contained therein can flow more easily and continuously to the bore and be extracted there. In fracking, a fluid (“fracfluid”) is pressed into the geological horizon, from which extraction is supposed to take place, at a high pressure of typically several hundred bar. The fracfluid is water to which in general proppants, such as quartz sand, and thickeners have been added.

At this point, it is explicitly pointed out that features of the solutions described above, in the Claims and/or in the Figures can also be combined, if desired, in order to achieve cumulated implementation of the explained features, effects and advantages.

All features disclosed in the application documents are claimed as essential for the invention provided that they are, individually or in combination, novel over the state of the art.

It is explicitly pointed out that within the framework of the present patent application, indefinite articles and numerals such as “one”, “two” etc. are normally to be understood as indicating a minimum, i. e. “at least one . . . ”, “at least two . . . ” etc., unless it becomes explicitly clear from the context or is obvious or imperative to the person skilled in the art from a technical point of view that only “exactly one . . . ”, “exactly two . . . ” etc. can be intended.

Other advantages, particularities and expedient embodiments of the invention will become clear from the dependent Claims and the following presentation of preferred embodiments by means of the Figures.

The embodiments shown here are only examples of the present invention and are therefore not to be understood as limiting. Alternative embodiments considered by the person skilled in the art are equally comprised by the scope of protection of the invention.

### EXAMPLES OF EMBODIMENT

1 Description of the Manufacturing Process on the Basis of a Rough Flow Diagram

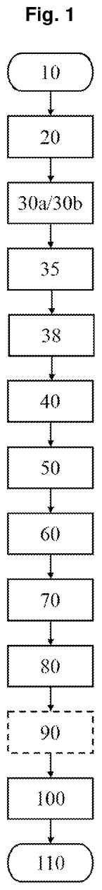

FIG. 1 is a schematic representation of a method of producing the citrus fiber in the form of a flowchart. Starting from the citrus pulp 10, the pulp is disintegrated by hydrolytic 30 incubation in an acidic solution at 70° to 80° C. Two separate steps 30a (decanter) and 30b (separator) follow for as complete a separation of all particles from the liquid phase as possible. The separated material is resuspended with an aqueous NaOH solution in step 35, wherein a suspension with a pH value of between 3.0 and 5.0 is obtained. In 38 coarse or non-disintegrated particles are separated through sieving by means of a straining machine. In step 40 the enzymatic de-esterification takes place by adding a pectin methylesterase and an incubation over 2 to 8 hours at 10 to 60° C. Then, two alcohol washing steps 50 and 70 are performed with subsequent solid-liquid separation by means of decanters 60 and 80. In step 100, finally, the fibers are gently dried by fluidized bed drying so as to obtain the citrus fibers 110 according to the invention.

2 Test Method for Determining the Yield Point (Rotational Measurement)

Measurement Principle:

This yield point is an indicator of the structural strength and is determined by rotational measurement, by increasing the shear stress acting on the sample over time until the sample begins to flow.

Shear stresses below the yield point merely cause an elastic deformation; it is only shear stresses above the yield point that will cause the sample to flow. This value is determined by measuring when a defined minimum shear rate y is exceeded. According to the present method, the yield point To [Pa] is exceeded at shear rate γ≥0.1 s⁻¹.
measuring device: Rheometer Physica MCR series (e.g. MCR 301, MCR 101)
measuring system: Z3 DIN or CC25, respectively
measuring vessel: CC 27 P06 (ribbed measuring vessel)
number of stages: 3
measuring temperature: 20° C.
measuring parameters:

Rheometer Physica MCR series

1^{st} Stage (Resting Period):

| | | |
|---|---|---|
| stage settings: | default parameter: | shearing stress [Pa] |
| | value: | 0 Pa constant |

| | | |
|---|---|---|
| stage duration: | 180 | s |
| temperature: | 20° | C. |

2^{nd} Stage (Determining of Yield Point):

| | | |
|---|---|---|
| stage settings: | default parameter: | shearing stress [Pa] |
| | profile: | ramp log. |

| | | |
|---|---|---|
| initial value: | 0.1 | Pa |
| final value: | 80 | Pa |
| stage duration: | 180 | s |
| temperature: | 20° | C. |

Evaluation:

The yield point To (unit [Pa]) is read out in stage 2 and is the shearing stress (unit: [Pa]) at which the shear rate is for the last time γ≥0.10 s⁻¹.

The yield point measured with the rotation method is also called “yield point (rotation)”.

The yield point (rotation) was measured using a fiber suspension (the fiber was simply stirred in with a spoon=corresponding to a non-activated fiber) and is also called “yield point (rotation) II” within the context of the invention. The yield point is also measured using a fiber dispersion (stirred in under the effect of high shearing forces, e. g. with Ultra Turrax=corresponding to an activated fiber) and is also called “yield point (rotation) I” within the context of the invention.

3 Test Method for Determining the Yield Point (Oscillation Measurement)

Measurement Principle:

This yield point is also an indicator of the structural strength and is determined in an oscillation test by increasing the amplitude at constant frequency until the sample is destroyed due to the ever-increasing excursion and starts to flow.

Below the yield point, the substance behaves like an elastic solid, i. e. the elastic portions (G′) amount to a larger portion than the viscous portions (G″) whereas when the yield point is exceeded, the viscous portions of the sample increase and the elastic portions decrease.

By definition, the yield point is exceeded at the amplitude when the amount of viscous portions equals that of elastic portions; G′=G″ (cross over); the corresponding shear stress is the respective measured value.
Measuring device: Rheometer Physica MCR series (e.g. MCR 301, MCR 101)
Measuring system: Z3 DIN or CC25
Measuring vessel: CC 27 P06 (ribbed measuring vessel)
Measuring Parameters:

| Stage settings: | amplitude defaults: | deformation [%] |
|---|---|---|
| | profile: | ramp log. |
| | value: | 0.01-1000% |

| | | |
|---|---|---|
| frequency: | 1.0 | Hz |
| temperature: | 20° | C. |

Evaluation:

By means of the rheometer software Rheoplus, the shear stress at cross over is evaluated after the linear viscoelastic range has been exceeded.

The yield point measured with the oscillation method is also called “yield point (cross over)”.

The yield point (cross over) was measured using a fiber suspension (the fiber was simply stirred in with a spoon=corresponding to a non-activated fiber) and is also termed “yield point (cross over) II” within the framework of the invention. The yield point was additionally measured using a fiber dispersion (stirred in under the effect of high shearing forces, e. g. with Ultra Turrax=corresponding to a shear-activated fiber) and is also called “yield point (cross over) I” in the context of the invention.

Measuring Results and Their Implications:

If the yield point of the suspensions of the fibers used according to the invention, stirred in with a spoon (corresponding to a non-shear-activated fiber), is compared to that of a fiber dispersion stirred in under high shearing forces, such as Ultra Turrax corresponding to an activated fiber), a statement on the advantage/necessity of an activation can be made. The measuring results are summarized in the table below. As can be expected, the yield point increases by shear activation in the dispersion. Due to the relatively low yield points of fiber suspensions To II, full implementation of fiber properties requires activation of the fiber to obtain the desired creamy texture.

| | rotation | | cross over | | |
|---|---|---|---|---|---|
| | τₒ II [Pa] | τₒ I [Pa] | τₒ II [Pa] | τₒ I [Pa] | activa- |
| fiber | suspension | dispersion | suspension | dispersion | tion |
| Activatable, | 0.8 | 3.0 | 0.6 | 3.4 | neces- |
| de-esterified | | | | | sary |
| citrus fiber | | | | | |
| used according | | | | | |
| to the invention | | | | | |

4 Test Method for Determining the Dynamic Weissenberg Number

Measuring Principle and Meaning of the Dynamic Weissenberg Number:

The dynamic Weissenberg number W′ (Windhab E, Maier T, Lebensmitteltechnik 1990, 44: 185f) is a derived variable in which the elastic portions (G′) determined in the linear viscoelastic range are related to the viscous portions (G″) in an oscillation test:

$${W}^{′} = \frac{{G}^{′} ( ω )}{{G}^{″} ( ω )} = \frac{1}{tan δ}$$

The dynamic Weissenberg number is a variable which correlates particularly well with the sensorial perception of consistency and can be regarded quite independently from the absolute firmness of the sample.

A high value of W′ means that the fibers have a predominantly elastic structure, whereas a low value of W′ indicates structures with clearly viscous portions. The creamy texture typical of fibers is achieved if the W′ values lie within a range of approximately 6-8; if the values are lower, the sample is assessed to be aqueous (thickened less strongly).

Material and Methods:
measuring device: Rheometer Physica MCR series, e.g. MCR 301, MCR 101
measuring system: Z3 DIN or CC25
measuring vessel: CC 27 P06 (ribbed measuring vessel)

Measuring Parameters:

| stage settings: | amplitude defaults: | deformation |
|---|---|---|
| | profile: | ramp log |
| | value: | 0.01-1000% |

| | | |
|---|---|---|
| frequency: | 1.0 | Hz |
| temperature: | 20° | C. |

Evaluation:

The angle of phase difference 5 is read out within the linear viscoelastic range. Subsequently, the dynamic Weissenberg number W′ is calculated with the following formula:

$${W}^{′} = \frac{1}{tan δ}$$

Measuring Results and Their Implications:

Comparing the dynamic Weissenberg number W′ for the suspension of a fiber used according to the invention, stirred in with a spoon (corresponding to a non-shear-activated fiber) with a fiber dispersion, stirred in with high shearing forces, e. g. Ultra Turrax (corresponding to an activated fiber), a statement on the texture and, in addition, on the necessity of an activation can be made. The measuring results are summarized in the table below. The citrus fiber used according to the invention is, with W′ values of 7.2 in the suspension and 7.5 for the dispersion, in the ideal range and thus has an optimum texture. The texture is in both cases creamy. Thus, the results concerning the dynamic Weissenberg number show that activation of the fiber is not absolutely necessary to obtain a desired creamy texture.

| | W′ | W′ | |
|---|---|---|---|
| fiber | suspension | dispersion | texture |
| Activatable, de- | 8.6 | 8.8 | creamy with and without |
| esterified citrus | | | activation, viscosity/ |
| fiber used according | | | yield point are regulated |
| to the invention | | | by dosing |

5 Testing Method for Determining Firmness

Method:

150 ml distilled water are introduced into a beaker. Then 6.0 g of citrus fibers or 9.0 g of apple fibers are stirred into the water with a spoon without formation of lumps. For swelling, this fiber-water mixture is left to stand for 20 min. The suspension is then transferred into a vessel (0 90 mm). Subsequently, the firmness is measured with the following method:
Measuring device: Texture Analyser TA-XT 2 (company Stable Micro Systems, Godalming, UK)

Testing method/option: measuring of force in the direction of pressure/simple test
Parameters:—testing speed: 1.0 mm/s
   —distance: 15.0 mm/s
Measuring tool: —P/50

The firmness corresponds to the force required by the measuring bob to penetrate into the suspension by 10 mm. This force is then read out from the force-time diagram. It is to note that due to the history of the firmness measurement the unit of measured firmness has manifested itself in grams (g).

6 Test Method for Determining the Particle Size

Measurement Principle:

In a sieving machine, a set of sieves with a mesh width continuously increasing from the lower sieve to the upper sieve is arranged on top of each other. The sample is placed on the top sieve—the one with the largest mesh width. The sample particles with a diameter larger than the mesh width remain on the sieve; the finer particles fall onto the sieve below it. The portion of sample on the various sieves is weighed and indicated as a percentage.

Method:

The sample is weighed in to the second decimal digit. The sieves are provided with sieving aids and stacked on top of each other with the mesh width increasing. The sample is quantitatively transferred onto the top sieve; the sieves are clamped in, and the sieving process is performed according to defined parameters. The individual sieves are weighed with the sample and the sieving aid as well as empty with the sieving aid. If for a product, only a limit value within the particle size spectrum is to be tested (e. g. 90%<250 μm), only a sieve with the respective mesh width is used.

Measuring Defaults:
sample amount: 15 g
sieving aids: 2 per sieve bottom
sieving machine: AS 200 digit, Retsch GmbH company
sieving movement: three-dimensional
oscillation height: 1.5 mm
sieving duration: 15 min

The sieve structure consists of the following mesh widths in μm: 1400, 1180, 1000, 710, 500, 355, 250, 150, followed by the bottom.

The particle size is calculated using the following formula:

$$portion per sieve in \% = \frac{final weight in g on the sieve \times 100}{initial sample weight in g}$$

7 Preparation of a 2.5 wt % Fiber Dispersion

Formula:
2.5 g fiber
97.5 g demineralized water (room temperature)
duration of sprinkling: 15 seconds

The respective amount of demineralized water (room temperature) is introduced into a 250 ml beaker. The exactly weighed amount of fiber is slowly and directly poured into the stirring maelstrom with the stirring unit (Ultra Turrax) running at 8000 rpm (level 1). The sprinkling duration depends on the amount of fibers; it is to last 15 seconds per 2.5 g of sample. Then the dispersion is stirred for exactly 60 seconds at 8000 rpm (level 1). If the sample is to be used for determining viscosity or for determining the yield point I (rotation), the yield point I (cross over) or for determining the dynamic Weissenberg number, it is placed in a temperature-controlled water bath at 20° C.

For measuring viscosity or for measuring the yield point I (rotation), the yield point I (cross over) or for measuring the dynamic Weissenberg number, the sample is carefully given, after exactly 1 hour, into the measurement system of the rheometer, and the respective measurement is started. If the sample settles, it is carefully stirred up by means of a spoon directly before bottling.

8 Preparation of a 2.5 wt % Fiber Suspension

Formula:
2.50 g fiber
97.5 g demineralized water (room temperature)

The respective amount of demineralized water (room temperature) is introduced into a 250 ml beaker. The exactly weighed amount of citrus fiber is slowly poured in with a plastic spoon under constant stirring. Then the suspension is stirred until all fibers are watered. If the sample is to be used for determining viscosity or for determining the yield point II (rotation), the yield point II (cross over) or for determining the dynamic Weissenberg number, it is placed in a temperature-controlled water bath at 20° C.

For measuring viscosity or for measuring the yield point II (rotation), the yield point II (cross over) or for measuring the dynamic Weissenberg number, the sample is carefully filled into the measurement system of the rheometer after exactly 1 hour, and the measurement is started. If the sample settles, it is carefully stirred up by means of a spoon directly before bottling.

9 Test Method for Determining Water Binding Capacity

Implementation for water binding capacity of non-pretreated samples:

The sample is left to swell over 24 hours with a water excess at room temperature. After centrifugation and subsequent decanting of the supernatant, the water binding capacity can be gravimetrically determined in g H₂O/g sample. The pH value in the suspension is to be measured and documented.

The following parameters are to be observed:

Weighed Portions:
plant fiber: 1.0 g (in centrifuge tube)
water addition: 60 ml
centrifugation: 4000 g
duration of centrifugation: 10 min

20 minutes after beginning of centrifugation (i. e. 10 minutes after the end of centrifugation), the water supernatant is separated from the welled sample. The sample with the bound water is weighed.

The water binding capacity (WBC) in g H₂O/g sample can now be calculated with the following formula:

$$WBC ( \frac{g H 2 O}{g sample} ) = \frac{sample with bound water [ g ] - 1. g}{1. g}$$

10 Testing Method for Determining Viscosity
measuring device: Physica MCR series (e. g. MCR 301, MCR 101)
measuring system: Z3 DIN or CC25 (note: the measuring systems Z3 DIN and CC25 are identical)
number of stages: 4

Measuring Parameters:

1^{st} Stage:

| | | |
|---|---|---|
| stage settings: | default parameter: | shearing speed [s⁻¹] |
| | profile: | constant |

| | | |
|---|---|---|
| value: | 0 | s⁻¹ |
| stage duration: | 60 | s |
| temperature: | 20° | C. |

2^{nd} Stage:

| | | |
|---|---|---|
| stage settings: | default parameter: | shearing speed [s⁻¹] |
| | profile: | ramp linear |

| | | |
|---|---|---|
| value: | 0.1-100 | s⁻¹ |
| stage duration: | 120 | s |
| temperature: | 20° | C. |

3^{rd} Stage:

| | | |
|---|---|---|
| stage settings: | default parameter: | shearing speed [s⁻¹] |
| | profile: | constant |

| | | |
|---|---|---|
| value: | 100 | s⁻¹ |
| stage duration: | 10 | s |
| temperature: | 20° | C. |

4^{th} Stage:

| | | |
|---|---|---|
| stage settings: | default parameter: | shearing speed [s⁻¹] |
| | profile: | ramp linear |

| | | |
|---|---|---|
| value: | 100-0.1 | s⁻¹ |
| stage duration: | 120 | s |
| temperature: | 20° | C. |

Evaluation:

The viscosity (unit [mPas]) is read out as follows: 4^{th} stage at=50 s⁻¹

11 Testing Method for Determining the Degree Of Esterification

This method corresponds to the method published by JECFA (Joint FAO/WHO Expert Committee on Food Additives). Other than in the JECFA method, however, the deashed pectin is not dissolved in the cold, but heated. Isopropanol instead of ethanol is used as the alcohol.

12 Testing Method for Determining the Dietary Fiber Content

This method substantially corresponds to the one published by the AOAC (Official Method 991.43: Total, Soluble and Insoluble Dietary Fiber in Foods; Enzymatic-Gravimetric Method, MES-TRIS Buffer, First Action 1991, Final Action 1994). The only difference is that isopropyl alcohol was used here instead of ethanol.

13 Testing Method for Determining Moisture and Dry Matter Content

Principle:

The moisture content of the sample is understood to be the reduction in mass after drying, determined according to defined preconditions. The moisture content is determined by means of infrared drying with the moisture analyzer Sartorius MA-45 (Sartorius company, Gottingen, Federal Republic of Germany).

Method:

Approximately 2.5 g of the fiber sample are weighed in on the Sartorius moisture analyzer. The settings of the device can be found in the respective factory measuring instructions. For measuring, the samples are to have approximately room temperature. The moisture content is automatically indicated in percent [% M] by the device. The content of dry substance is automatically indicated in percent [96 S] by the device.

14 Testing Method for Determining Color and Lightness

Principle:

The measurements of color and lightness are performed with the Minolta Chromameter CR 300 or CR 400, respectively. The spectral characteristics of a sample are determined using tristimulus values. The color of a sample is described using the hue, the lightness and saturation. With these three basic properties, the color can be represented three-dimensionally.

The hues are located on the external face of the color solid; the lightness changes on the vertical axis and the degree of saturation horizontally. If the L*a*b* measurement system is employed, L* represents lightness whereas a* and b* indicate both the hue and the saturation. a* and b* indicate the positions on two color axes, with a* being assigned to the red-green axis and b* being assigned to the blue-yellow axis. For indicating the color measurement values, the device converts the tristimulus values into L*a*b* coordinates.

Performance of Measurement:

The sample is sprinkled on a white sheet of paper and flattened with a glass plug. For measurement, the measuring head of the chromameter is directly placed on the sample and the trigger is actuated. A triple measurement is performed of each sample and the average value calculated. The L*, a* and b* values are indicated by the device with two decimals.

The embodiments shown here are only examples of the present invention and are therefore not to be understood as limiting. Alternative embodiments considered by the person skilled in the art are equally comprised by the scope of protection of the present invention.

15 Testing Method for Determining Water-Soluble Pectin in Fiber-Containing Samples

Principle:

By aqueous extraction, the pectin contained in fiber-containing samples is converted into the liquid phase. By the addition of alcohol, the pectin is precipitated from the extract as an alcohol-insoluble substance (AIS).

Extraction:

10 g of the sample to be examined is weighed into a glass bowl. 390 g of boiling distilled water is introduced into a beaker, and the previously weighed sample is stirred into it for 1 minute at the maximum level with Ultra-Turrax.

The sample suspension cooled to ambient temperature is divided over four 150 ml-centrifuge beakers and centrifuged over 10 min at 4000×g. The supernatant is collected. The sediment of each beaker is re-suspended with 50 g of distilled water and again centrifuged over 10 min at 4000×g. The supernatant is collected, the sediment discarded.

The combined centrifugates are added to approximately 4 l of isopropanol (98%) for precipitation of the alcohol-insoluble substance (AIS). After ½ hour, filtration takes place by a filter cloth and the AIS is manually pressed out. In the filter cloth, the AIS is then added to approximately 3 1 of isopropanol (98%) and manually, with use of gloves, loosened.

The pressing procedure is repeated, the AIS quantitatively removed from the filter cloth, loosened and dried at 60° C. in a drying oven for 1 hour.

0.1g of the dried substance which has been pressed out is weighed for calculation of the alcohol-insoluble substance (AIS).

Calculation:

Calculation of the water-soluble pectin, with reference to the fiber-containing sample, is performed according to the following formula, with the water-soluble pectin being precipitated as the alcohol-insoluble substance (AIS):

$$AIS in the sample in wt \% ( \frac{g}{100 g} ) = \frac{dried AIS [ g ] \times 100}{initial sample weight in g}$$

### LIST OF REFERENCE NUMBERS

10 citrus pulp

20 hydrolysis by incubation in an acidic environment

30a 1^{st} solid-liquid separation decanter

30b 2^{nd} solid-liquid separation separator

35 resuspension with diluted NaOHaq solution

38 separation of coarse and non-disintegrated particles

40 enzymatic de-esterification

50 1^{st} washing with alcohol

60 solid-liquid separation

70 2^{nd} washing with alcohol

80 solid-liquid separation

100 fluidized bed drying

110 obtained de-esterified citrus fiber

1. A method of preparing a product comprising providing an activatable, de-esterified fruit fiber, wherein the product is selected from the group consisting of food products, feeding stuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, characterized in that the activatable, de-esterified fruit fiber has a content of water-soluble pectin of 10 wt % or less.

2. A method of preparing a product comprising providing an activatable, de-esterified fruit fiber, wherein the product is in the construction area, in extraction by drilling boreholes and in agriculture, characterized in that the activatable, de-esterified fruit fiber has a content of water-soluble pectin of 10 wt % or less.

3. The method according to claim 1, characterized in that the activatable, de-esterified fruit fiber has one or more of the following functions: as a foaming agent, a whipping agent, a release agent, a free flow agent, a stabilizer, an emulsifier, a carrier, a filler, a texturing agent, a thickener, a gelling agent, a solidifying agent, a dietary fiber, a reinforcing agent, a humectant, a filter aid, an egg substitute, a glazing agent, an improving agent for freeze-thaw stability and an improving agent for baking stability.

4. The method according to claim 1, characterized in that the activatable, de-esterified fruit fiber has a content of water-soluble pectin of 10 wt % or less and the pectin is a low methoxyl pectin and wherein the fruit fiber is preferably an activatable, de-esterified citrus fiber or an activatable, de-esterified apple fiber.

5. The method according to claim 1, characterized in that the activatable, de-esterified fruit fiber is an activatable, de-esterified citrus fiber which has one or more of the following properties:
a. a yield point II (rotation) in the fiber suspension of 0.1-1.5 Pa, advantageously of 0.4-1.0 Pa, and particularly advantageously of 0.6-0.8 Pa;
b. a yield point II (cross over) in the fiber suspension of 0.1-1.0 Pa, advantageously of 0.2-0.7 Pa, and particularly advantageously of 0.3-0.5 Pa;
c. a yield point I (rotation) in the fiber dispersion of 3.0-7.0 Pa, advantageously of 4.0-5.5 Pa, and particularly advantageously of 4.3-5.3 Pa;
d. a yield point I (cross over) in the fiber dispersion of 4.0-7.0 Pa, advantageously of 4.5-6.5 Pa, and particularly advantageously of 5.0-6.0 Pa;
e. a dynamic Weissenberg number in the fiber suspension of 7.0-10.0, advantageously of 7.5-9.5, and particularly advantageously of 8.1-9.1;
f. a dynamic Weissenberg number in the fiber dispersion of 7.5-10.0, advantageously of 8.0-9.5 and particularly advantageously of 8.3-9.3;
g. a firmness in a 4 wt % aqueous suspension of between 60 g and 240 g, preferably of between 120 g and 200 g, and particularly preferably of between 140 g and 180 g;
h. a viscosity of 550 to 850 mPas, preferably of 600 to 800 mPas, and particularly preferably of 650 to 750 mPas, wherein the de-esterified citrus fiber is dispersed in water as a 2.5 wt % solution and the viscosity is measured with a shear rate of 50 s−1 at 20° C.;
i. a water binding capacity of more than 24 g/g, preferably of more than 26 g/g, particularly preferably of more than 28 g/g, and especially preferably of between 28 and 32 g/g;
j. a moisture of less than 15%, preferably of less than 10% and particularly preferably of less than 8%;
k. in a 1.0 wt % aqueous solution, a pH value of 5.0 to 6.0 and preferably of 5.2 to 5.7;
l. a particle size wherein at least 90% of the particles are smaller than 450 μm, preferably at least 90% of the particles are smaller than 350 μm and particularly preferably at least 90% of the particles are smaller than 250 μm;
m. a lightness value L*>84, preferably L*>86 and particularly preferably L*>88;
n. a fiber content of the activatable, de-esterified citrus fiber of 80 to 95%;
o. the activatable, de-esterified citrus fiber comprises a content of water-soluble pectin of 10 wt % or less, preferably between 2 and 8 wt % and particularly preferably of between 2 and 6 wt %.

6. The method according to claim 1, characterized in that the activatable, de-esterified fruit fiber is an activatable, de-esterified apple fiber which has one or more of the following properties:
a. a yield point II (rotation) in the fiber suspension of 0.1-1.5 Pa, advantageously of 0.4-1.0 Pa, and particularly advantageously of 0.6-0.8 Pa;
b. a yield point II (cross over) in the fiber suspension of 0.1-1.0 Pa, advantageously of 0.2-0.7 Pa, and particularly advantageously of 0.3-0.5 Pa;
c. a yield point I (rotation) in the fiber dispersion of 3.0-7.0 Pa, advantageously of 4.0-5.5 Pa, and particularly advantageously of 4.3-5.3 Pa;
d. a yield point I (cross over) in the fiber dispersion of 4.0-7.0 Pa, advantageously of 4.5-6.5 Pa, and particularly advantageously of 5.0-6.0 Pa;
e. a dynamic Weissenberg number in the fiber suspension of 7.0-10.0, advantageously of 7.5-9.5, and particularly advantageously of 8.1-9.1;
f. a dynamic Weissenberg number in the fiber dispersion of 7.5-10.0, advantageously of 8.0-9.5 and particularly advantageously of 8.3-9.3;
g. a firmness in a 4 wt % aqueous suspension of between 60 g and 240 g, preferably of between 120 g and 200 g, and particularly preferably of between 140 g and 180 g;
h. a viscosity of 550 to 850 mPas, preferably of 600 to 800 mPas, and particularly preferably of 650 to 750 mPas, wherein the de-esterified apple fiber is dispersed in water as a 2.5 wt % solution and the viscosity is measured with a shear rate of 50 s−1 at 20° C.;
i. a water binding capacity of more than 24 g/g, preferably of more than 26 g/g, particularly preferably of more than 28 g/g, and especially preferably of between 28 and 32 g/g;
j. a moisture of less than 15%, preferably of less than 10% and particularly preferably of less than 8%;
k. in a 1.0 wt % aqueous solution, a pH value of 5.0 to 6.0 and preferably of 5.2 to 5.7;
l. a particle size wherein at least 90% of the particles are smaller than 450 μm, preferably at least 90% of the particles are smaller than 350 μm and particularly preferably at least 90% of the particles are smaller than 250 μm;
m. a lightness value L*>60, preferably L*>61 and particularly preferably L*>62;
n. a fiber content of the activatable, de-esterified apple fiber of 80 to 95%;
o. the activatable, de-esterified apple fiber comprises a content of water-soluble pectin of 10 wt % or less, preferably between 2 and 8 wt % and particularly preferably of between 2 and 6 wt %.

7. The method according to claim 1, characterized in that the food product is selected from the group consisting of preserved products, deep-frozen foods, vegan food, vegetarian food, gluten-free food, low-calorie food, low-sugar food, lactose-free food, jelly, jelly-type sweets, sauce, granola bars, fruit pieces, fruit snacks, fruit bars, milk substitute drink, milk substitute product, foam sugar products, sherbet, ice cream, desserts, fermented drink, milk product, delicacies, fruit drink, fruit drink containing alcohol, cocktail, vegetable drink, chutney, barbecue sauce, smoothies, instant drink, fruit spread, fruit compote, fruit dessert, fruit sauce, fruit preparations, bake-stable fruit preparations, fruit preparations for yoghurt, bake-stable vegetable preparations, bake-stable fatty fillings, baked goods, pasta and pasta fillings, noodle dishes, potato snack, cheese and cream cheese preparations, meat substitute products, extruder products, corn flakes, breakfast cereals, soup, sauce, mayonnaise, meats, sausages, sausage casing, seafood, spirits, lozenges, functional food, nutritional supplements and dietary foods such as enteral foods, dysphagia food or sip feed.

8. The method Use according to claim 1, characterized in that the feeding stuff is selected from the group consisting of feeding stuff rich in starches, oleaginous feedstuff, feedstuff rich in protein, extrudate feedstuff, wet feed, binder, bird feedstuff rod, rodent feedstuff rod, fish bait, supplement feedstuff, feedstuff for particular nutritional purposes and dietary feedstuff.

9. The method according to claim 1, characterized in that the animal need is animal bedding.

10. The method according to claim 1, characterized in that the hygiene product is selected from the group consisting of diapers, incontinence articles like protective trousers or incontinence pants, sanitary towels, tampons, panty liners and softcups.

11. The method according to claim 1, characterized in that the personal care product is selected from the group consisting of soap, shower gel, bath additives, skin creams, lotions, gel, sunscreen, sun cream, repellent, shaving cream, shaving soap, epilator cream, toothpaste, dentition adhesive medium, shampoo, hair forming agents, hair-setting products, hair colorants, facial make-up, eye care products, lip care products, nail polish and self-tanning agents.

12. The method according to claim 1, characterized in that the cleaning agent is selected from the group consisting of detergent, bile soap, washing-up liquid, dishwasher detergent, rinsing agent, neutral cleaner, abrasive cleaner, window cleaner, lime remover, drain cleaner, brake cleaner, alcohol cleaner, all-purpose cleaner, glass cleaner, sanitary cleaner, toilet cleaner, toilet gel, toilet soap, carpet cleaner, car care material, oven cleaner, bathroom cleaner and metal polish, oil absorbing and anti-dust agent.

13. The method according to claim 1, characterized in that the coating agent is selected from the group consisting of antistatic coating, oil-repellent coating and anti-block coating.

14. The method according to claim 1, characterized in that the explosive is a gelatinous explosive.

15. The method according to claim 1, characterized in that the lubricant is selected from the group consisting of liquid lubricant, such as lubricating oil and cooling lubricant, lubricating grease and solid lubricant.

16. The method according to claim 1, characterized in that the plastic product is a citrus fiber-reinforced plastic or a wood-plastic composite (WPC).

17. The method according to claim 1, characterized in that the varnish is selected from the group consisting of alkyd resin varnish, oil varnish, cellulose nitrate varnish, bitumen varnish, tar-containing varnish, phenolic resin varnish, urea resin varnish, melamine resin varnish, polyester varnish, epoxy resin varnish, polyurethane resin varnish, acrylic varnish and powder varnish.

18. The method according to claim 1, characterized in that the coating agent is selected from the group consisting of glaze, oil paint, dispersion paint, chalk paint, silicate paint and liquid plaster.

19. The method according to claim 1, characterized in that the construction material is selected from the group consisting of building foam, sound proofing material, insulation material, concrete, screed, mortar, cement, chemical bonded anchors, chemical anchor bolts, asphalt and whisper asphalt.

20. The method according to claim 1, characterized in that the adhesive is selected from the group consisting of dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive, paste.

21. The method according to claim 1, characterized in that the fertilizer is a binder for fertilizer cones.

22. The method according to claim 1, characterized in that the drug is selected from the group consisting of powder, juice, lotion, ointment, cream, gel, tablet and rubber article.

23. The method according to claim 1, characterized in that the medical product is selected from the group consisting of wound dressing, emergency bandage, transdermal patch, stoma product and dental casting compound.

24. The method according to claim 1, characterized in that the battery is selected from the group consisting of primary cell, accumulator and solid cell battery.

25. The method according to claim 2, characterized in that use in the construction area comprises usage in road and path construction, masonry construction, concrete construction and reinforced concrete construction.

26. The method according to claim 2, characterized in that the use in extraction by drilling boreholes comprises the usage as an additive to a drilling fluid or fracfluid.

27. The method according to claim 2, characterized in that the use in agriculture comprises the usage in fertilizers, humectants, soil conditioners, plant substrates, flowerpots and substrate-tablet extrudates.

28. Product selected from the group consisting of food products, feeding stuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, characterized in that it comprises an activatable, de-esterified fruit fiber according to claim 4.

29. Product according to claim 28, wherein the portion of activatable, de-esterified fruit fiber is between 0.05 wt % and 90 wt %, preferably between 0.1 and 50 wt %, particularly preferably from 0.1 to 25 wt % and especially preferably between 0.5 and 10 wt %.
